(19) United States
(12) Patent Application Publication
(10) Pub. No.: US 2014/0148578 A1
(43) Pub. Date: May 29, 2014

(54) BLENDED SOY PROTEIN PRODUCTS HAVING ALTERED CHARACTERISTICS

(71) Applicants: Phillip S. Kerr, Wildwood, MO (US); Naina K. Shah, Manchester, MO (US); Anthony J Irwin, MO (CA); Daniel U Staerk, Kirkwood, MO (US); Nicolas Deak, University, MO (US)

(72) Inventors: Phillip S. Kerr, Wildwood, MO (US); Naina K. Shah, Manchester, MO (US); Anthony J Irwin, MO (CA); Daniel U Staerk, Kirkwood, MO (US); Nicolas Deak, University, MO (US)

(73) Assignee: EI DU PONT DE NEMOURS AND COMPANY, Wilmington (DE)

(21) Appl. No.: 13/810,014

(22) PCT Filed: Jul. 14, 2011
(86) PCT No.: PCT/US11/44025

§ 371 Date: May 24, 2013

Related U.S. Application Data
(60) Provisional application No. 61/364,045, filed on Jul. 14, 2010.

(51) Int. Cl. C07K 14/415

(52) U.S. Cl. CPC C07K 14/415
USPC 530/378

(57) ABSTRACT

Blended soy protein products with improved whiteness, altered viscosity and altered gel-strength, are described. Use of such products in food, beverage and animal feed are also disclosed.

### FIELD OF THE INVENTION

This invention relates to blended soy protein products with improved whiteness, altered viscosity and altered gel-strength.

### BACKGROUND OF THE INVENTION

Soybeans have the highest protein content of all cereals and legumes. In particular, soybeans have about 40% protein, while other legumes have 20-30%, and cereals have about 8-15% protein. Soybeans also contain about 20% oil with the remaining dry matter mostly carbohydrate (35%). On a wet basis (as is), soybeans contain about 35% protein, 17% oil, 31% carbohydrates and 4.4% ash. Soybean storage protein and lipid bodies are contained in the usable meat of the soybean called the cotyledon. The complex carbohydrate (or dietary fiber) is also contained in the cell walls of the cotyledon. The outer layer of cells (called the seed coat) makes up about 8% of the soybean's total weight. The raw, dehulled soybean is, depending on the variety, approximately 18% oil, 15% insoluble carbohydrates, 14% moisture and ash and 38% protein.

Plant protein materials are used as functional food ingredients, and have numerous applications in enhancing desirable characteristics in food products. Soy protein materials, in particular, have seen extensive use as functional food ingredients. Soy protein materials are used as an emulsifier in meats to bind the meat and give the meat a good texture and a firm bite. Another common application for soy protein materials as functional food ingredients is as a thickening agent to provide a creamy viscosity to the food product.

In general, soy protein materials include soy flakes, soy grits, soy meal, soy flour, soy protein concentrates, and soy protein isolates with a primary difference between these materials being the degree of refinement relative to whole soybeans.

Apart from the soy protein content, flavor, gel-strength, whiteness-index, and viscosity of a soy protein material are also a relevant criteria for the selection of a soy protein material as a functional food ingredient. Conventional soy protein material may have a strong beany, bitter flavor and odor as a result of the presence of certain volatile compounds and/or an undesired appearance due to the presence of other relatively low molecular weight compounds in the soy protein material.

The present disclosure generally relates to a blended soy protein product-containing composition having altered (e.g. increased or reduced) gel-strength, viscosity, or whiteness.

U.S. Pat. No. 6,599,556 B2, issued to Stark et al. on Jul. 29, 2003, describes confectionery products, which include high protein content modified oilseed material.

U.S. Pat. No. 6,716,469 B2, issued to Stark et al. on Apr. 6, 2004, describes frozen dessert products, which include high protein content modified oilseed material.

U.S. Pat. No. 6,720,020 B2, issued to Karleskind et al. on Apr. 13, 2004, describes beverage compositions, which include high protein content modified oilseed material.

JP Patent No. 5,168,416 A1, issued to Takeshi et al. on Jul. 2, 1993, describes obtaining a concentrated soybean having improved taste, flavor and color tone and useful as a food material, etc., with simple operation at a low cost without changing the nature of the protein by washing soybeans, etc., with a water-containing alcohol under weakly acidic condition in the presence of an acid.

JP Patent No. 4,207,159 A1, issued to Hiroko et al on Jul. 29, 1992, describes the title raw material having bright and white color tone and useful for marine and knead eater—dispersed liquid of acid-precipitated soybean protein with an alkali metal hydroxide to control pH.

WO2007013146A1, published Feb. 1, 2007, describes compositions for processed soy protein foods.

WO2009086047A2, published Jul. 9, 2009, describes soybean protein products having altered characteristics.

### SUMMARY OF THE INVENTION

In a first embodiment, the invention concerns a blended soy protein product, comprising a soy protein product obtained from commodity soybean defatted flakes and at least one other different soybean defatted flake with a combined PUFA content of about 0.17% to about 1.65% by weight. In another embodiment, the blended soy protein product is a blend of high oleic soy flakes and commodity soy flakes.

In a second embodiment, the invention concerns a blended soy protein product comprising a first soy protein product obtained from commodity soybean defatted flake and at least one other different soybean defatted flake with a combined PUFA content of about 0.17% to about 1.65% by weight, wherein the first product, has reduced viscosity when compared to a second soy protein product obtained from the commodity soybean defatted flakes, and further wherein the same process is used to produce the first and second protein product.

In a third embodiment, the invention concerns a first soy protein product obtained from commodity soybean defatted flakes and at least one other different soybean defatted flake with a combined PUFA content of about 0.17% to about 1.65% by weight, wherein the first product, has improved drying efficiency when compared to a second soy protein product obtained from the commodity soybean defatted flakes, and further wherein the same process is used to produce the first and second protein product.

In a fourth embodiment, the invention concerns a first soy protein product obtained from commodity soybean defatted flake and at least one other different soybean defatted flakes with a combined PUFA content of about 0.17% to about 1.65% by weight, wherein said first product, has an improved whiteness index when compared to a second soy protein product obtained from the commodity soybean defatted flakes, and further wherein the same process is used to produce the first and second protein product.

In a fifth embodiment, the invention concerns a blended soy protein product comprising a first soy protein product obtained from commodity soybean defatted flake and at least one other different soybean defatted flake with a combined PUFA content of about 0.17% to about 1.65% by weight, wherein the viscosity of the first soy protein product is reduced by at least 25% when compared to a second soy protein product obtained from the commodity soybean defatted flakes, and further wherein the same process is used to produce the first and second protein product.

In a sixth embodiment, the invention concerns a first soy protein product obtained from commodity soybean defatted flakes and at least one other different soybean defatted flake with a combined PUFA content of about 0.17% to about 1.65% by weight, wherein the drying efficiency of the first soy protein product is increased by at least 1% when compared to a second soy protein product obtained from the commodity soybean defatted flakes, and further wherein the same process is used to produce the first and second protein product.

In a seventh embodiment, the invention concerns a first soy protein product obtained from commodity soybean defatted flakes and at least one other different soybean defatted flake with a combined PUFA content of about 0.17% to about 1.65% by weight, wherein the whiteness index of the first soy protein product is increased by at least 2.5% when compared to a second soy protein product obtained from the commodity soybean detailed flakes, and further wherein the same process is used to produce the first and second protein product.

In an eighth embodiment, the invention concerns a method for improving drying efficiency of a soy protein product, comprising:

a) feeding a first soy protein product obtained from commodity soybean defatted flakes and at least one other different soybean defatted flake at higher feed solids to a pasteurizer or a dryer, compared to feeding a second soy protein product obtained from the commodity soybean defatted flakes; and

b) measuring improved drying efficiency of the soy protein product.

In a ninth embodiment, the invention concerns a method of preparing a soy protein product with an increased whiteness index, comprising:

a) obtaining defatted flakes from a first commodity soybean and from a second soybean with a combined PUFA content of about 0.17% to about 1.65% by weight; and

b) measuring increased whiteness.

In a tenth embodiment, the invention concerns a method of preparing a soy protein product with reduced viscosity, comprising:

a) obtaining defatted flakes from a first commodity soybean and from a second soybean with a combined PUFA content of about 0.17% to about 1.65%; and

b) measuring reduced viscosity.

In an eleventh embodiment, the invention concerns a first soy protein product obtained from commodity soybean defatted flakes and at least one other different soybean defatted flake with a combined PUFA content of more than 1.65% by weight, wherein said first product, has increased viscosity when compared to a second soy protein product obtained from the other different soybean defatted flakes, and further wherein the same process is used to produce the first and second protein product.

In another embodiment, the invention concerns a first soy protein product obtained from commodity soybean defatted flakes and at least one other different soybean defatted flake with a combined PUFA content of more than 1.65% by weight, wherein said first product, has increased gel strength when compared to a second soy protein product obtained from the other different soybean defatted flakes, and further wherein the same process is used to produce the first and second protein product.

In yet another embodiment, the invention concerns a first soy protein product obtained from high oleic soybean defatted flakes, obtained by the method comprising of preparing defatted flakes from a high oleic soybean; adding PUFAs; and measuring the viscosity of the soy protein product; wherein said first soy protein product has increased viscosity when compared to a second soy protein product obtained from high oleic soybean defatted flakes without addition of PUFAs, and further wherein the same process is used to produce the first and second protein product. In some embodiments the added PUFA content is about 0.066% to about 3.85% by weight.

In another embodiment the invention concerns soy protein products selected from the group consisting of a soy protein isolate, a soy protein concentrate, soy meal, full fat flour, defatted flour, soymilk powder, soymilk, textured proteins, textured flours, textured concentrates and textured isolates.

Additional embodiments of the invention include soy protein products with at least 40%, 65%, or 90% protein (N×6.25) on a moisture-free basis.

In other aspects, the soy protein products of the invention can be used in food, beverages, and animal feed containing the soy protein product of the invention.

### BRIEF DESCRIPTION OF THE DRAWINGS AND SEQUENCE LISTINGS

The invention can be more fully understood from the following detailed description and the accompanying drawings and Sequence Listing, which form a part of this application.

FIG. 1 depicts plasmid pKS210.

FIG. 2 depicts plasmid PHP17731.

FIG. 3 depicts plasmid PHP17064.

FIG. 4 depicts fragment PHP19340A.

FIG. 5 depicts fragment PHP17752A.

FIG. 6 depicts plasmid PHP19340.

FIG. 7 depicts plasmid PHP17752.

FIG. 8 shows the effect of mixing high oleic and commodity flakes on the resultant isolate slurrys' in-process apparent viscosity as a function of shear.

FIG. 9 shows the effect of PUFA content of the high oleic and commodity flake mix on the in-process apparent viscosity

FIG. 10 shows the effect of PUFA addition on the whiteness index of the freeze dried soy protein isolate powder.

FIG. 11 shows the effect of individual fatty acids on the viscosity of the soy protein products of the invention.

SEQ ID NO:1 sets forth the sequence of the recombinant DNA fragment PHP21676A

SEQ ID NO:2 sets forth the sequence of the 1533 polynucleotide fragment comprising 470 nucleotides from the soybean FAD2-2 gene, 420 nucleotides from the soybean FAD2-1 gene, 643 nucleotides from the soybean FAD3 gene.

SEQ ID NO:3 sets forth the nucleotide sequence of oligonucleotide primer BM35 used to amplify an approximately 0.9 Kb fragment from recombinant DNA fragment KSFAD2-hybrid.

SEQ ID NO:4 sets forth the nucleotide sequence of oligonucleotide primer BM39 used to amplify an approximately 0.9 kb fragment from recombinant DNA fragment KSFAD2-hybrid.

SEQ ID NO:5 sets forth the nucleotide sequence of oligonucleotide primer BM40 used to amplify an approximately 0.65 kb DNA fragment from plasmid XF1.

SEQ ID NO:6 sets forth the nucleotide sequence of oligonucleotide plasmid BM41 used to amplify an approximately 0.65 kb DNA fragment from plasmid pXF1.

SEQ ID NO:7 sets forth the nucleotide sequence of recombinant DNA fragment KSFAD2-hybrid which contains about 470 nucleotides from the soybean FAD2-2 gene and 420 nucleotides from the soybean FAD2-1 gene.

SEQ ID NO:8 sets forth the nucleotide sequence of oligonucleotide primer KS1 used to amplify about 470 nucleotides from the soybean FAD2-2 gene.

SEQ ID NO:9 sets forth the nucleotide sequence of oligonucleotide primer KS2 used to amplify about 470 nucleotides of the soybean FAD2-2 gene.

SEQ ID NO:10 sets forth the nucleotide sequence of oligonucleotide primer KS3 used to amplify about 420 nucleotides of the soybean FAD2-1 gene.

SEQ ID NO:11 sets forth the nucleotide sequence of oligonucleotide primer KS4 used to amplify about 420 nucleotides of the soybean FAD2-1 gene.

SEQ ID NO:12 sets forth the nucleotide sequence of the seed-specific gene expression-silencing cassette from pKS133 which comprises nucleotides for a Kti3 promoter and terminator bordering a string of nucleotides that promote formation of a stem structure which are surrounding a unique Not I restriction endonuclease site.

SEQ ID NO:13 sets forth the nucleotide sequence of plasmid pKS210. SEQ ID NO:14 sets forth the nucleotide sequence of plasmid PHP17731.

SEQ ID NO:15 sets forth the nucleotide sequence of recombinant DNA fragment PHP17731A.

SEQ ID NO:16 sets forth the nucleotide sequence of the ALS selectable marker recombinant DNA fragment. This recombinant DNA fragment comprises a promoter operably linked to a nucleotide fragment encoding a soybean acetolactate synthase to which mutations have been introduced to make it resistant to treatment with sulfonylurea herbicides.

SEQ ID NO:17 sets forth the amino acid sequence of the soybean herbicide-resistant ALS including mutations in subsequences B and F.

SEQ ID NO:18 is the wild type amino acid sequence of conserved ALS “subsequence B” disclosed in U.S. Pat. No. 5,013,659.

SEQ ID NO:19 sets forth the wild type amino acid sequence of conserved ALS “subsequence F” disclosed in U.S. Pat. No. 5,013,659.

SEQ ID NO:20 sets forth the amino acid sequence of the additional five amino acids introduced during cloning at the amino-terminus of the soybean ALS.

SEQ ID NO:21 sets forth the nucleotide sequence of plasmid PHP17064

SEQ ID NO:22 sets forth the nucleotide sequence of recombinant DNA fragment PHP17064A.

SEQ ID NO:23 sets forth the nucleotide sequence of fragment PHP19340A.

SEQ ID NO:24 sets forth the nucleotide sequence of fragment PHP17752A.

SEQ ID NO:25 sets forth the nucleotide sequence of plasmid PHP19340.

SEQ ID NO:26 sets forth the nucleotide sequence of plasmid PHP17752.

The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (No. 2):345-373 (1984) which are herein incorporated by reference. The symbols and format used for nucleotide and amino acid sequence data comply with the rules set forth in 37 C.F.R. §1.822.

### DETAILED DESCRIPTION OF THE INVENTION

All patents, patent applications, and publications cited herein are incorporated by reference in their entirety.

The present disclosure generally relates to a blended soy protein product-containing composition having altered (e.g. increased or reduced) gel-strength, viscosity, or whiteness.

In the context of this disclosure, a number of terms shall be utilized.

As used herein, “soybean” refers to the species Glycine max, Glycine soja, or any species that is sexually cross compatible with Glycine max. A “line” is a group of plants of similar parentage that display little or no genetic variation between individuals for a least one trait. Such lines may be created by one or more generations of self-pollination and selection, or vegetative propagation from a single parent including by tissue or cell culture techniques. An “agronomically elite line” or “elite line” refers to a line with desirable agronomic performance that may or may not be used commercially. A “variety”, “cultivar”, “elite variety”, or “elite cultivar” refers to an agronomically superior elite line that has been extensively tested and is or was being used for commercial soybean production. “Mutation” refers to a detectable and heritable genetic change (either spontaneous or induced) not caused by segregation or genetic recombination. “Mutant” refers to an individual, or lineage of individuals, possessing a mutation.

The “whiteness index” of a soy protein product refers to the color of the soy-protein-containing composition. Many soy protein-containing feed compositions will have, to varying degrees, a yellowish or brownish color. In general, the color of these compositions can be “improved,” i.e., the “whiteness index” of the product can be increased by the process of the present invention. In general, the whiteness index is determined using a colorimeter which provides the L, a, and b color values for the composition from which the whiteness index may be calculated using a standard expression of the Whiteness Index (WI), WI=L−3b. The L component generally indicates the whiteness or, “lightness”, of the sample; L values near 0 indicate a black sample while L values near 100 indicate a white sample. The b value indicates yellow and blue colors present in the sample; positive b values indicate the presence of yellow colors while negative b values indicate the presence of blue colors. The a value, which may be used in other color measurements, indicates red and green colors; positive values indicate the presence of red colors while negative values indicate the presence of green colors. For the b and a values, the absolute value of the measurement increases directly as the intensity of the corresponding color increases. Generally, the colorimeter is standardized using a white standard tile provided with the colorimeter. A sample is then placed into a glass cell which is introduced to the colorimeter. The sample cell is covered with an opaque cover to minimize the possibility of ambient light reaching the detector through the sample and serves as a constant during measurement of the sample. After the reading is taken, the sample cell is emptied and typically refilled as multiple samples of the same material are generally measured and the whiteness index of the material expressed as the average of the measurements. Suitable colorimeters generally include those manufactured by HunterLab (Reston, Va.) including, for example, Model # DP-9000 with Optical Sensor D 25.

Whiteness index measurements of a 5% by weight solids sample of the suspension before and after treatment are determined using a HunterLab DP-9000 colorimeter including an optical sensor D-25, both manufactured by Hunter Associates Laboratory (HunterLab) (Reston, Va.). For the whiteness index measurement in the large scale production platform, protein samples are dispersed on a 5% w/w basis: (5.25 g) is added to deionized water (100 mL). For the whiteness index measurement in the small scale production platform, 1 g protein sample is dispersed in 19 mL of deionized water on a w/v basis. The results obtained using the Hunter Colorimeter are reported in units of L, a, and b. Whiteness Index is calculated from the L and b scale values using the following: Whiteness Index=L−3b.

In addition to the improved color, the soy protein product produced by the processes in the present disclosure can have a reduced viscosity.

Viscosity, gelation and other indicators of structure formation are important properties of soybean proteins since they contribute to the overall utility of the product in use. Proteins contribute to the solidity and elasticity of products by formation of a three dimensional network of aggregated protein molecules which entrap water. It is sometimes desirable to have these properties, for example in the case of meat-like products, or it may be desired to have less functionality, for example in beverage applications. For beverage applications, a lower viscosity may be desirable for sensory, mouthfeel and textural properties of the beverage. Lower viscosity soy protein-containing compositions may be intended for use in liquid products (i.e., beverages); and additionally, in some embodiments, higher viscosity soy protein-containing compositions may be desired for use in meat products.

As used herein, the term “viscosity” means the apparent viscosity of aqueous slurry or a solution as measured with a rotating spindle viscometer utilizing a large annulus, where a particularly preferred rotating spindly viscometer is an Anton Paar MCR-300. The apparent viscosity can also be measured using a Rapid Visco Analyzer (RVA) viscometer, Brookfield viscometer, or other viscometers and rheometers known in the art.

The apparent viscosity of a soy protein material may be measured, for example, by weighing a sample of the soy material and water to obtain a known ratio of the soy material to water (preferably 1 part soy material to 9 parts water, by weight), combining and mixing the soy material and water in a blender or mixer to form a homogenous slurry of the soy material and water at ambient temperature and neutral pH, and measuring the apparent viscosity of the slurry with the rotating spindle viscometer utilizing a large annulus, operated at approximately 60 revolutions per minute and at a torque of from 30 to 70%.

Another important functional characteristic is the gel forming property of a protein. Protein gelation is important to obtain desirable sensory and textural structures in foods.

The formation of a protein gel is a two step process which initiates through partial denaturation of the protein molecules. As the proteins denature, the viscosity of the slurry increases as a result of an increase in the molecular changes associated with the unfolding proteins. During the second part of the process there is a large increase in viscosity resulting from protein association and development of the molecular network.

Gelation phenomenon requires a driving force to unfold the native protein structure, followed by an aggregation retaining a certain degree of order in the matrix formed by association between protein strands. Protein gelation has been traditionally achieved by heating, but some physical and chemical processes form protein gels in an analogous way to heat-induction. A physical means, besides heat, is high pressure. Chemical means are acidification, enzymatic cross-linking, and use of salts and urea, causing modifications in protein-protein and protein-medium interactions. The characteristics of each gel are different and dependent upon factors like protein concentration, degree of denaturation caused by pH, temperature, ionic strength and/or pressure.

The term “gel-strength” refers to the ability or a measure of a protein to form gels.

“Polyunsaturated fatty acid(s) and their triglycerides” are abbreviated PUFA(s).

The term “fatty acids” refers to long-chain aliphatic acids (alkanoic acids) of varying chain length, from about C₁₂ to C₂₂ (although both longer and shorter chain-length acids are known). The predominant chain lengths are between C₁₆ and C₂₂. The structure of a fatty acid is represented by a simple notation system of “X:Y”, where X is the total number of C atoms in the particular fatty acid and Y is the number of double bonds.

Generally, fatty acids are classified as saturated or unsaturated. The term “saturated fatty acids” refers to those fatty acids that have no “double bonds” between their carbon backbone. In contrast, “unsaturated fatty acids” have “double bonds” along their carbon backbones (which are most commonly in the cis-configuration). “Monounsaturated fatty acids” have only one “double bond” along the carbon backbone (e.g., usually between the 9th and 10th carbon atom as for palmitoleic acid (16:1) and oleic acid (18:1)), while “polyunsaturated fatty acids” (or “PUFAs”) have at least two double bonds along the carbon backbone (e.g., between the 9th and 10th, and 12th and 13th carbon atoms for linoleic acid (18:2); and between the 9^{th} and 10^{th}, 12^{th} and 13^{th}, and 15^{th} and 16^{th} for α-linolenic add (18:3)).

The term “total fatty acid content” refers to the sum of the five major fatty acid components found in soybeans, namely C16:0, C18:0, C18:1, C18:2, and C18:3. The term “total polyunsaturated fatty acid content” refers to the total C18:2 plus C18:3 content.

For the purposes of the present disclosure, the omega-reference system will be used to indicate the number of carbons, the number of double bonds and the position of the double bond closest to the omega carbon, counting from the omega carbon (which is the terminal carbon of the aliphatic chain and is numbered 1 for this purpose). This nomenclature is shown below in Table 1, in the column titled “Shorthand Notation”.

TABLE 1
Nomencature of Polyunsaturated Fatty Acids

| | | | Shorthand |
|---|---|---|---|
| Common Name | Abbreviation | Chemical Name | Notation |
| Linoleic | LA | cis-9,12-octadecadienoic | 18:2 ω-6 |
| α-Linolenic | αLIN | cis-9,12,15- | 18:3 ω-3 |
| | | octadecatrienoic | |

The term “desaturase” refers to a polypeptide that can desaturate, i.e., introduce a double bond, in one or more fatty acids to produce a mono- or polyunsaturated fatty acid or precursor which is of interest. Despite use of the omega-reference system throughout the specification in reference to specific fatty acids, it is more convenient to indicate the activity of a desaturase by counting from the carboxyl end of the substrate using the Δ-system.

The terms “FAD” and fatty acid desaturase are used interchangeably and refer to membrane bound microsomal oleoyl- and linoleoyl-phosphatidylcholine desaturases that convert oleic acid to linoleic acid and linoleic acid to linolenic acid, respectively, in reactions that reduce molecular oxygen to water and require the presence of NADH.

The term “high oleic soybean” refers to soybean seeds that have an oleic acid content of at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, and 95% of the fatty acid moieties. Preferred high oleic soybean oil starting materials are disclosed in World Patent Publication WO94/11516, the disclosure of which is hereby incorporated by reference.

Some embodiments of the instant invention comprise soybean seeds with an oleic acid content of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, and 95% of the fatty acid moieties.

The term enzyme “activity” refers to the ability of an enzyme to convert a substrate to a product.

The terms “polynucleotide”, “polynucleotide sequence”, “nucleic acid sequence”, “nucleic acid fragment”, and “isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof. Nucleotides (usually found in their 5ζ-monophosphate form) are referred to by a single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

The terms “subfragment that is functionally equivalent” and “functionally equivalent subfragment” are used interchangeably herein. These terms refer to a portion or subsequence of an isolated nucleic acid fragment in which the ability to alter gene expression or produce a certain phenotype is retained whether or not the fragment or subfragment encodes an active enzyme. For example, the fragment or subfragment can be used in the design of chimeric genes to produce the desired phenotype in a transformed plant.

Chimeric genes can be designed for use in suppression by linking a nucleic acid fragment or subfragment thereof, whether or not it encodes an active enzyme, in the sense or antisense orientation relative to a plant promoter sequence.

The terms “homology”, “homologous”, “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant invention such as deletion or insertion of one or more nucleotides that do not substantially alter the functional properties of the resulting nucleic acid fragment relative to the initial, unmodified fragment. It is therefore understood, as those skilled in the art will appreciate, that the invention encompasses more than the specific exemplary sequences.

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure. An “allele” is one of several alternative forms of a gene occupying a given locus on a chromosome. When all the alleles present at a given locus on a chromosome are the same that plant is homozygous at that locus. If the alleles present at a given locus on a chromosome differ that plant is heterozygous at that locus. A “codon-optimized gene” is a gene having its frequency of codon usage designed to mimic the frequency of preferred codon usage of the host cell.

“Coding sequence” refers to a DNA sequence that codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences.

“Promoter” refers to a region of DNA capable of controlling the expression of a coding sequence or functional RNA. The promoter sequence consists of proximal and more distal upstream elements. These upstream elements are often referred to as enhancers. Accordingly, an “enhancer” is a DNA sequence that can stimulate promoter activity, and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity. Promoters that cause a gene to be expressed in most cell types at most times are commonly referred to as “constitutive promoters”. New promoters of various types useful in plant cells are constantly being discovered; numerous examples may be found in the compilation by Okamuro and Goldberg (1989) Biochemistry of Plants 15:1-82.

Any seed-specific promoter can be used in accordance with the method of the invention. Thus, the origin of the promoter chosen to drive expression of the recombinant DNA fragment is not critical as long as it is capable of accomplishing the invention by transcribing enough RNA from the desired nucleic acid fragment(s) in the seed.

A plethora of promoters is described in WO 00/18963, published on Apr. 6, 2000, the disclosure of which is hereby incorporated by reference. Examples of seed-specific promoters include, and are not limited to, the promoter for soybean Kuntz trypsin inhibitor (Kti3, Jofuku and Goldberg (1989) Plant Cell 1:1079-1093) β-conglycinin (Chen et al. (1989) Dev. Genet. 10: 112-122), the napin promoter, and the phaseolin promoter.

Specific examples of promoters that may be useful in expressing the nucleic acid fragments of the invention include, but are not limited to, the SAM synthetase promoter (PCT Publication WO00/37662, published Jun. 29, 2000), the CaMV 35S (Odell at al (1985) Nature 313:810-812), and the promoter described in PCT Publication WO02/099063 published Dec. 12, 2002.

The “translation leader sequence” refers to a polynucleotide sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences have been described (Turner and Foster (1995) Mol. Biotechnol. 3:225-236).

The “3′ non-coding sequences” or “transcription terminator/termination sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al. (1989) Plant Cell 1:671-680.

An “Intron” is an intervening sequence in a gene that does not encode a portion of the protein sequence. Thus, such sequences are transcribed into RNA but are then excised and are not translated. The term is also used for the excised RNA sequences. An “axon” is a portion of the sequence of a gene that is transcribed and is found in the mature messenger RNA derived from the gene, but is not necessarily a part of the sequence that encodes the final gene product.

“RNA transcript” refers to the product resulting from RNA polymerase-catalyzed transcription of a DNA sequence. When the RNA transcript is a perfect complementary copy of the DNA sequence, it is referred to as the primary transcript. An RNA transcript is referred to as the mature RNA when it is an RNA sequence derived from post-transcriptional processing of the primary transcript. “Messenger RNA (mRNA)” refers to the RNA that is without introns and that can be translated into protein by the cell. “cDNA” refers to a DNA that is complementary to and synthesized from a mRNA template using the enzyme reverse transcriptase. The cDNA can be single-stranded or converted into the double-stranded form using the Klenow fragment of DNA polymerase I. “Sense” RNA refers to RNA transcript that includes the mRNA and can be translated into protein within a cell or in vitro. “Antisense RNA” refers to an RNA transcript that is complementary to all or part of a target primary transcript or mRNA, and that blocks the expression of a target gene (U.S. Pat. No. 5,107,065). The complementarity of an antisense RNA may be with any part of the specific gene transcript, i.e., at the 5′ non-coding sequence, 3 non-coding sequence, introns, or the coding sequence. “Functional RNA” refers to antisense RNA, ribozyme RNA, or other RNA that may not be translated but yet has an effect on cellular processes. The terms “complement” and “reverse complement” are used interchangeably herein with respect to mRNA transcripts, and are meant to define the antisense RNA of the message.

The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another example, the complementary RNA regions of the invention can be operably linked, either directly or indirectly, 5′ to the target mRNA, or 3′ to the target mRNA, or within the target mRNA, or a first complementary region is 5′ and its complement is 3′ to the target mRNA.

The term “endogenous RNA” refers to any RNA which is encoded by any nucleic acid sequence present in the genome of the host prior to transformation with the recombinant construct of the present invention, whether naturally-occurring or non-naturally occurring, i.e., introduced by recombinant means, mutagenesis, etc.

The term “non-naturally occurring” means artificial, not consistent with what is normally found in nature.

Standard recombinant DNA and molecular cloning techniques used herein are well known in the art and are described more fully in Sambrook et al., Molecular Cloning: A Laboratory Manual; Cold Spring Harbor Laboratory Press: Cold Spring Harbor, 1989. Transformation methods are well known to those skilled in the art and are described below.

“PCR” or “Polymerase Chain Reaction” is a technique for the synthesis of large quantities of specific DNA segments, consists of a series of repetitive cycles (Perkin Elmer Cetus instruments, Norwalk, Conn.). Typically, the double stranded DNA is heat denatured, the two primers complementary to the 3′ boundaries of the target segment are annealed at low temperature and then extended at an intermediate temperature. One set of these three consecutive steps is referred to as a cycle.

The term “recombinant” refers to an artificial combination of two otherwise separated segments of sequence, e.g., by chemical synthesis or by the manipulation of isolated segments of nucleic acids by genetic engineering techniques.

The terms “plasmid”, “vector” and “cassette” refer to an extra chromosomal element often carrying genes that are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitates transformation of a particular host cell.

“Expression cassette” refers to a specific vector containing a foreign gene and having elements in addition to the foreign gene that allow for enhanced expression of that gene in a foreign host.

The terms “recombinant construct”, “expression construct”, “chimeric construct”, “construct”, and “recombinant DNA construct” are used interchangeably herein. A recombinant construct comprises an artificial combination of nucleic acid fragments, e.g., regulatory and coding sequences that are not found together in nature. For example, a chimeric construct may comprise regulatory sequences and coding sequences that are derived from different sources, or regulatory sequences and coding sequences derived from the same source, but arranged in a manner different than that found in nature. Such construct may be used by itself or may be used in conjunction with a vector. If a vector is used then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells comprising any of the isolated nucleic acid fragments of the invention. The skilled artisan will also recognize that different independent transformation events will result in different levels and patterns of expression (Jones et al., (1985) EMBO J. 4:2411-2418; De Almeida et al., (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, immunoblotting analysis of protein expression, or phenotypic analysis, among others.

The term “expression”, as used herein, refers to the production of a functional end-product e.g., a mRNA or a protein (precursor or mature).

The term “expression cassette” as used herein, refers to a discrete nucleic acid fragment into which a nucleic acid sequence or fragment can be moved.

“Mature” protein refers to a post-translationally processed polypeptide; i.e., one from which any pre- or propeptides present in the primary translation product have been removed. “Precursor” protein refers to the primary product of translation of mRNA; i.e., with pre- and propeptides still present. Pre- and propeptides may be but are not limited to intracellular localization signals.

“Cosuppression” refers to the production of sense RNA transcripts capable of suppressing the expression of identical or substantially similar native genes (U.S. Pat. No. 5,231,020, which issued to Jorgensen et al. on Jul. 27, 1999). Co-suppression constructs in plants have been previously designed by focusing on overexpression of a nucleic acid sequence having homology to a native mRNA, in the sense orientation, which results in the reduction of all RNA having homology to the overexpressed sequence (see Vaucheret et al. (1998) Plant J. 16:651-659; and Gura (2000) Nature 404:804-808). “Antisense inhibition” refers to the production of antisense RNA transcripts capable of suppressing the expression of the target protein. Plant viral sequences may be used to direct the suppression of proximal mRNA encoding sequences (POT Publication WO 98/36083 published on Aug. 20, 1998). “Hairpin” structures that incorporate all, or part, of an mRNA encoding sequence in a complementary orientation resulting in a potential “stem-loop” structure for the expressed RNA have been described (POT Publication WO 99/53050 published on Oct. 21, 1999). In this case the stem is formed by polynucleotides corresponding to the gene of interest inserted in either sense or anti-sense orientation with respect to the promoter and the loop is formed by some polynucleotides of the gene of interest, which do not have a complement in the construct. This increases the frequency of cosuppression or silencing in the recovered transgenic plants. For review of hairpin suppression see Wesley at al. (2003) Methods in Molecular Biology, Plant Functional Genomics; Methods and Protocols 236:273-286. A construct where the stem is formed by at least 30 nucleotides from a gene to be suppressed and the loop is formed by a random nucleotide sequence has also effectively been used for suppression (WO 99/61632 published on Dec. 2, 1999). The use of poly-T and poly-A sequences to generate the stem in the stem-loop structure has also been described (WO 02/00894 published Jan. 3, 2002). Yet another variation includes using synthetic repeats to promote formation of a stem in the stem-loop structure. Transgenic organisms prepared with such recombinant DNA fragment show reduced levels of the protein encoded by the polynucleotide from which the nucleotide fragment forming the loop is derived as described in POT Publication WO 02/00904, published Jan. 3, 2002. The use of constructs that result in dsRNA has also been described. In these constructs convergent promoters direct transcription of gene-specific sense and antisense RNAs inducing gene suppression (see for example Shi et al. (2000) RNA 6:1069-1076; Bastin et al. (2000) J. Cell Sci. 113:3321-3328; Giordano et al. (2002) Genetics 160:637-648; LaCount; and Donelson US patent Application No. 20020182223, published Dec. 5, 2002; Tran et al. (2003) BMC Biotechnol. 3:21; and Applicant's U.S. Provisional Application No. 60/578,404, filed Jun. 9, 2004).

Other methods for suppressing an enzyme include, but are not limited to, use of polynucleotides that may form a catalytic RNA or may have ribozyme activity (U.S. Pat. No. 4,987,071 issued Jan. 22, 1991), and micro RNA (also called miRNA) interference (Javier et al. (2003) Nature 425:257-263),

MicroRNAs (miRNA) are small regulatory RNAs that control gene expression. miRNAs bind to regions of target RNAs and inhibit their translation and, thus, interfere with production of the polypeptide encoded by the target RNA. miRNAs can be designed to be complementary to any region of the target sequence RNA including the 3′ untranslated region, coding region, etc. miRNAs are processed from highly structured RNA precursors that are processed by the action of a ribonuclease III termed DICER. While the exact mechanism of action of miRNAs is unknown, it appears that they function to regulate expression of the target gene. See, e.g., U.S. Patent Publication No. 2004/0268441 A1 which was published on Dec. 30, 2004.

The term “expression”, as used herein, refers to the production of a functional end-product, be it mRNA or translation of mRNA into a polypeptide.

“Antisense inhibition” refers to the production of antisense RNA transcripts capable of suppressing the expression of the target protein. “Co-suppression” refers to the production of sense RNA transcripts capable of suppressing the expression of identical or substantially similar foreign or endogenous genes (U.S. Pat. No. 5,231,020).

“Overexpression” refers to the production of a functional end-product in transgenic organisms that exceeds levels of production when compared to expression of that functional end-product in a normal, wild type or non-transformed organism.

“Stable transformation” refers to the transfer of a nucleic acid fragment into a genome of a host organism, including both nuclear and organellar genomes, resulting in genetically stable inheritance. In contrast, “transient transformation” refers to the transfer of a nucleic acid fragment into the nucleus, or DNA-containing organelle, of a host organism resulting in gene expression without integration or stable inheritance. Host organisms containing the transformed nucleic acid fragments are referred to as “transgenic” organisms.

Standard recombinant DNA and molecular cloning techniques used herein are well known in the art and are described by Sambrook et al., Molecular Cloning: A Laboratory Manual, 2nd ed., Cold Spring Harbor Laboratory: Cold Spring Harbor, N.Y. (1989); by Silhavy et al., Experiments with Gene Fusions, Cold Spring Harbor Laboratory: Cold Spring Harbor, N.Y. (1984); and by Ausubel et al., Current Protocols in Molecular Biology, published by Greene Publishing Assoc. and Wiley-Interscience (1987). Once the recombinant construct has been made, it may then be introduced into a plant cell or yeast cell of choice by methods well known to those of ordinary skill in the art including, for example, transfection, transformation and electroporation (see below). Oilseed plant cells are the preferred plant cells. The transformed plant cell is then cultured and regenerated under suitable conditions permitting expression of the recombinant construct which is then recovered and purified.

Recombinant constructs may be introduced into one plant cell or, alternatively, a construct may be introduced into separate plant cells.

Expression in a plant cell may be accomplished in a transient or stable fashion as is described above.

Plant parts include differentiated and undifferentiated tissues, including but not limited to: roots, stems, shoots, leaves, pollen, seeds, tumor tissue, and various forms of cells and culture such as single cells, protoplasts, embryos, and callus tissue. The plant tissue may be in plant or in organ, tissue or cell culture.

The term “plant organ” refers to plant tissue or group of tissues that constitute a morphologically and functionally distinct part of a plant. The term “genome” refers to the following: 1. The entire complement of genetic material (genes and non-coding sequences) is present in each cell of an organism, or virus or organelle. 2. A complete set of chromosomes inherited as a (haploid) unit from one parent. The term “stably integrated” refers to the transfer of a nucleic acid fragment into the genome of a host organism or cell resulting in genetically stable inheritance.

Methods for transforming dicots, primarily by use of Agrobacterium tumefaciens, and obtaining transgenic plants have been published, among others, for cotton (U.S. Pat. No. 5,004,863, U.S. Pat. No. 5,159,135); soybean (U.S. Pat. No. 5,569,834, U.S. Pat. No. 5,416,011); Brass/ca (U.S. Pat. No. 5,463,174); peanut (Cheng et al. (1996) Plant Cell Rep. 15:653-657, McKently et al. (1995) Plant Cell Rep. 14:699-703); papaya (Ling et al. (1991) Bio/technology 9:752-758); and pea (Grant et al. (1995) Plant Cell Rep. 15:254-258). For a review of other commonly used methods of plant transformation see Newell (2000) Mol. Biotechnol, 16:53-65. One of these methods of transformation uses Agrobacterium rhizogenes (Tepfler, and Casse-Delbart (1987) Microbiol. Sci. 4:24-28). Transformation of soybeans using direct delivery of DNA has been published using PEG fusion (PCT publication WO 92/17598), electroporation (Chowrira et al. (1995) Mol. Biotechnol. 3:17-23; Christou et al. (1987) Proc. Natl. Acad. Sci, U.S.A. 84:3962-3966), microinjection, or particle bombardment (McCabe et. al. (1988) Bio/Technology 6:923; Christou et al. (1988) Plant Physiol. 87:671-674).

There are a variety of methods for the regeneration of plants from plant tissue. The particular method of regeneration will depend on the starting plant tissue and the particular plant species to be regenerated. The regeneration, development and cultivation of plants from single plant protoplast transformants or from various transformed explants is well known in the art (Weissbach and Weissbach, (1988) In.: Methods for Plant Molecular Biology, (Eds.), Academic: San Diego, Calif.). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present invention containing a desired polypeptide is cultivated using methods well known to one skilled in the art.

In addition to the above discussed procedures, practitioners are familiar with the standard resource materials which describe specific conditions and procedures for the construction, manipulation and isolation of macromolecules (e.g., DNA molecules, plasmids, etc.), generation of recombinant DNA fragments and recombinant expression constructs and the screening and isolating of clones, (see for example, Sambrook et al. (1989) Molecular Cloning: A Laboratory Manual, Cold Spring Harbor: NY; Maliga et al. (1995) Methods in Plant Molecular Biology, Cold Spring Harbor: NY; Birren et al. (1998) Genome Analysis: Detecting Genes, 1, Cold Spring Harbor: NY; Birren et al. (1998) Genome Analysis: Analyzing DNA, 2, Cold Spring Harbor: NY; Plant Molecular Biology: A Laboratory Manual, eds. Clark, Springer: NY (1997)).

In one aspect, the present invention includes protein products derived from high oleic soybeans.

The present invention includes a first (blended) protein product obtained from commodity soybean defatted flakes and at least one other different soybean defatted flake with a combined PUFA content of about 0.17% to about 1.65% by weight, wherein said first product, has at least one characteristic selected from the group of reduced viscosity, improved drying efficiency and improved whiteness index, when compared to a second soy protein product obtained from the commodity soybean defatted flakes, and further wherein the same process is used to produce the first and second protein product.

The various soy protein products disclosed in the invention can be blended with other protein products, vegetable oils, PUFAs or combinations thereof to alter or improve the protein products to suit desirable characteristics, such as, but not limited to, fatty acid composition, flavor, color, viscosity, gel strength or drying efficiency. The amount of other protein products, vegetable oils, PUFAs or combinations thereof which can be used depends on the desired functionality of the final product.

The invention additionally includes a first soy protein product obtained from high oleic soybean defatted flakes, obtained by the method comprising of preparing defatted flakes from a high oleic soybean; adding PUFAs; and measuring the viscosity of the soy protein product; wherein said first soy protein product has increased viscosity when compared to a second soy protein product obtained from high oleic soybean defatted flakes without addition of PUFAs, and further wherein the same process is used to produce the first and second protein product. In some embodiments the added PUFA content is about 0.066% to about 3.85% by weight of the flake.

Useful examples of the PUFA content (percent weight) of the various protein products of the invention, either added or present in native form or a combination thereof are 0.05%, 0.051%, 0.052%, 0.053%, 0.054%, 0.055, 0.056%, 0.057%, 0.058%, 0.059%, 0.06%, 0.061%, 0.062%, 0.063%, 0.064%, 0.065, 0.066%, 0.067%, 0.068%, 0.069%, 0.07%, 0.071%, 0.072%, 0.073%, 0.074%, 0.075, 0.076%, 0.077%, 0.078%, 0.079%, 0.08%, 0.081%, 0.082%, 0.083%, 0.084%, 0.085, 0.086%, 0.087%, 0.088%, 0.089%, 0.09%, 0.091%, 0.092%, 0.093%, 0.094%, 0.095, 0.096%, 0.097%, 0.098%, 0.099%, 0.1%, 0.11%, 0.12%, 0.13%, 0.14%, 0.15, 0.16%, 0.17%, 0.18%, 0.19%, 0.2%, 0.21%, 0.22%, 0.23%, 0.24%, 0.25, 0.26%, 0.27%, 0.28%, 0.29%, 0.3%, 0.31%, 0.32%, 0.33%, 0.34%, 0.35, 0.36%, 0.37%, 0.38%, 0.39%, 0.4%, 0.41%, 0.42%, 0.43%, 0.44%, 0.45, 0.46%, 0.47%, 0.48%, 0.49%, 0.5%, 0.51%, 0.52%, 0.53%, 0.54%, 0.55, 0.56%, 0.57%, 0.58%, 0.59%, 0.6%, 0.61%, 0.62%, 0.63%, 0.64%, 0.65, 0.66%, 0.67%, 0.68%, 0.69%_{;} 0.7%, 0.71%, 0.72%, 0.73%, 0.74%, 0.75, 0.76%, 0.77%, 0.78%, 0.79%, 0.8%, 0.81%, 0.82%, 0.83%, 0.84%, 0.85, 0.86%, 0.87%, 0.88%, 0.89%, 0.9%, 0.91%, 0.92%, 0.93%, 0.94%, 0.95, 0.96%, 0.97%, 0.98%, 0.99%, 1%, 1.01%, 1.02%, 1.03%, 1.04%, 1.05%, 1.06%, 1.07%, 1.08%, 1.09%, 1.1%, 1.11%, 1.12%, 1.13%, 1.14%, 1.15%, 1.16%, 1.17%, 1.18%, 1.19%, 1.2%, 1.21%, 1.22%, 1.23%, 1.24%, 1.25%, 1.26%, 1.27%, 1.28%, 1.29%, 1.3%, 1.31%, 1.32%, 1.33%, 1.34%, 1.35%, 1.36%, 1.37%, 1.38%, 1.39%, 1.4%, 1.41%, 1.42%, 1.43%, 1.44%, 1.45%, 1.46%, 1.47%, 1.48%, 1.49%, 1.5%, 1.51%, 1.52%, 1.53%, 1.54%, 1.55%, 1.56%, 1.57%, 1.58%, 1.59%, 1.6%, 1.61%, 1.62%, 1.63%, 1.64%, 1.65%, 1.66%, 1.67%, 1.68%, 1.69%, 1.7%, 1.71%, 1.72%, 1.73%, 1.74%, 1.75%, 1.76%, 1.77%, 1.78%, 1.79%, 1.8%, 1.81%, 1.82%, 1.83%, 1.84%, 1.85%, 1.86%, 1.87%, 1.88%, 1.89%, 1.9%, 1.91%, 1.92%, 1.93%, 1.94%, 1.95%, 1.96%, 1.97%, 1.98%, 1.99%, 2%, 2.01%, 2.02%, 2.03%, 2.04%, 2.05%, 2.06%, 2.07%, 2.08%, 2.09%, 2.1%, 2.11%, 2.12%, 2.13%, 2.14%, 2.15%, 2.16%, 2.17%, 2.18%, 2.19%, 2.2%, 2.21%, 2.22%, 2.23%, 2.24%, 2.25%, 2.26%, 2.27%, 2.28%, 2.29%, 2.3%, 2.31%, 2.32%, 2.33%, 2.34%, 2.35%, 2.36%, 2.37%, 2.38%, 2.39%, 2.4%, 2.41%, 2.42%, 2.43%, 2.44%, 2.45%, 2.46%, 2.47%, 2.48%, 2.49%, 2.5%, 2.51%, 2.52%, 2.53%, 2.54%, 2.55%, 2.56%, 2.57%, 2.58%, 2.59%, 2.6%, 2.61%, 2.62%, 2.63%, 2.64%, 2.65%, 2.66%, 2.67%, 2.68%, 2.69%, 2.7%, 2.71%, 2.72%, 2.73%, 2.74%, 2.75%, 2.76%, 2.77%, 2.78%, 2.79%, 2.8%, 2.81%, 2.82%, 2.83%, 2.84%, 2.85%, 2.86%, 2.87%, 2.88%, 2.89%, 2.9%, 2.91%, 2.92%, 2.93%, 2.94%, 2.95%, 2.96%, 2.97%, 2.98%, 2.99%, 3%, 3.01%, 3.02%, 3.03%, 3.04%, 3.05%, 3.06%, 3.07%, 3.08%, 3.09%, 3.1%, 3.11%, 3.12%, 3.13%, 3.14%, 3.15%, 3.16%, 3.17%, 3.18%, 3.19%, 3.2%, 3.21%, 3.22%, 3.23%, 3.24%, 3.25%, 3.26%, 3.27%, 3.28%, 3.29%, 3.3%, 3.31%, 3.32%, 3.33%, 3.34%, 3.35%, 3.36%, 3.37%, 3.38%, 3.39%, 3.4%, 3.41%, 3.42%, 3.43%, 3.44%, 3.45%, 3.46%, 3.47%, 3.48%, 3.49%, 3.5%, 3.51%, 3.52%, 3.53%, 3.54%, 3.55%, 3.56%, 3.57%, 3.58%, 3.59%, 3.6%, 3.61%, 3.62%, 3.63%, 3.64%, 3.65%, 3.66%, 3.67%, 3.68%, 3.69%, 3.7%, 3.71%, 3.72%, 3.73%, 3.74%, 3.75%, 3.76%, 3.77%, 3.78%, 3.79%, 3.8%, 3.81%, 3.82%, 3.83%, 3.84%, 3.85%, 3.86%, 3.87%, 3.88%, 3.89%, 3.9%, 3.91%, 3.92%, 3.93%, 3.94%, 3.95%, 3.96%, 3.97%, 3.98%, 3.99%. The amount and range of PUFAS which can be used will depend upon the desired properties sought to be achieved in the resulting final blended protein product. One of a preferred PUFA content range is of about 0.17% to about 1.65% by weight.

When an amount, concentration, or other value or parameter is given as either a range, preferred range or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

When the term “about” is used in describing a value or an end-point of a range, the disclosure should be understood to include the specific value or end-point referred to.

Another embodiment of the present invention concerns a first soy protein product obtained from commodity soybean defatted flakes and at least one other different soybean defatted flake with a combined PUFA content of about 0.17% to about 1.65% by weight, wherein said first product, has reduced viscosity of at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 58%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, or 87% when compared to a second soy protein product obtained from the commodity soybean defatted flakes, and further wherein the same process is used to produce the first and second protein product.

Another embodiment concerns a blended protein product comprising a first soy protein product obtained from commodity soybean defatted flakes and at least one other different soybean defatted flake with a combined PUFA content of about 0.17% to about 1.65% by weight, wherein the whiteness index is increased by at least 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, 30%, 30.5%, 31%, 31.5%, 32%, 32.5%, 33%, 315%_{;} 34%, 34.5%, 35%, 35.5%, 36%, 36.5%, 37%, 37.5%, 38%, 38.5%, 39%, 39.5%, 40%, 40.5%, 41%, 41.5%, 42%, 42.5%, 43%, 44%, 44.5%, 45%, 45.5%, 46%, 46.5%, 47%, 47.5%, 48%, 48.5%, 49%, 49.5% or 50% when compared to a second soy protein product obtained from the commodity soybean defatted flakes, and further wherein the same process is used to produce the first and second protein product.

Yet another embodiment concerns a blended protein product comprising a first soy protein product obtained from commodity soybean defatted flakes and at least one other different soybean defatted flake with a combined PUFA content of about 0.17% to about 1.65% by weight, wherein said first product, has an increased drying efficiency of at least 1%, 1.5%, 2%, 2.5%%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, and 30% when compared to a second soy protein product obtained from the commodity soybean defatted flakes, and further wherein the same process is used to produce the first and second protein product.

A first soy protein product obtained from commodity soybean defatted flakes and at least one other different soybean defatted flake with a combined PUFA content of about 0.17% to about 1.65% by weight, wherein said first product, has reduced gel strength when compared to a second soy protein product obtained from the other different soybean defatted flakes, and further wherein the same process is used to produce the first and second protein product.

A first soy protein product obtained from a commodity soybean defatted flake and at least one other different soybean detailed flake with a combined PUFA content of more than 1.65% by weight, wherein said first product, has increased gel strength when compared to a second soy protein product obtained from the other different soybean defatted flakes, and further wherein the same process is used to produce the first and second protein product.

Useful examples of altered (either reduced or increased) gel strength of the protein product of the invention are alterations in gel strength by at least 25%, 26%, 27%, 28%, 29%, 30%, 31%, 32%, 33%, 34%, 35%, 36%, 37%, 38%, 39%. 40%, 41%, 42%, 43%, 44%, 45%, 46%, 47%, 48%, 49%, 50%, 51%, 52%, 53%, 54%, 55%, 56%, 57%, 58%, 59%, or 60%.

One advantage to having reduced viscosity is that it improves drying efficiency. Currently with commodity soy there is a limitation in the feed solids concentration that can be fed to the dryer as a result of the viscosity and propensity to aggregate and form a gel as a result of exposure to heat. If commodity soy must be dried at high solids, it is necessary to increase the temperature of the feed solids to prevent protein aggregation with resultant gelling; this increased heat is costly and results in severe damage to the solubility of the protein.

Reduced viscosity and gel properties allow the operator to significantly increase the feed solid concentration, because the slurry can be easily pumped through the equipment at normal temperatures without gelling. That means that during the drying process, less water has to be removed for every pound fed to the dryer. This translates into decreased energy usage and more solids that can be dried per hour resulting in more protein product for sale.

Another embodiment of the invention concerns a soy protein product selected from the group consisting of a soy protein isolate, a soy protein concentrate, soy meal, full fat flour, defatted flour, soymilk, textured proteins, textured flours, textured concentrates and textured isolates.

As used herein, “soymilk” refers to an aqueous mixture of any one or more of the following, finely ground soybeans, soy flour, soy flakes, soy concentrate, isolated soy protein, soy whey protein, and aqueous extracts of any one or more of the following, soybeans, soy flakes and soy flour where insoluble material has been removed. Soymilk may comprise additional components including but not limited to fats, carbohydrates, sweeteners, colorants, stabilizers, thickeners, flavorings, acids, bases.

As used herein, “soymilk powder” refers to a dewatered soymilk. Soymilk may be dewatered by many processes that include but are not limited to spray drying, tray drying, tunnel drying, and freeze drying.

Another embodiment of the invention concerns a method for improving drying efficiency of a soy protein product, comprising feeding at least one soy protein product obtained from a high oleic soybean seed at higher feed solids to a pasteurizer or a dryer compared to feeding at least one soy protein product obtained from a commodity soybean to a pasteurizer or dryer.

An additional embodiment of the invention concerns a method for improving drying efficiency, comprising feeding a first blended soy protein product to a pasteurizer or a dryer at no less than 10%, 11%, 12.13%, 14%, 15%, 15%, 17%, 18%, 19%, 20%, 21%, 22%, 23%, 24%, 25%, 26%, 27%, 28%, 29%, or 30% feed solids compared to feeding commodity soy protein products using the same process as that to obtain the soy protein product from a high oleic soybean.

Soy protein products fall into three major groups. These groups are based on protein content, and range from 40% to over 90%. All three basic soy protein product groups (except full-fat flours) are derived from defatted flakes. They are the following: soy flours and grits, soy protein concentrates and soy protein isolates. These are discussed more fully below.

As used herein the term “unhydrolyzed protein product”, “unhydrolyzed soy protein product” refers to a protein product that has not undergone an enzymatic protein hydrolysis step.

As used herein the term “enzymatic hydrolysis” refers to the breakdown of proteins or chemical compounds by the addition of specific enzymes.

Additional embodiments of the invention include blended soy protein products with at least 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%,%, 89%, 90%, 91%, 92%, 93%, 94%, 95%, 96% or 97% protein (N×6.25) on a moisture-free basis.

The soy protein products of the invention can be incorporated into food, beverages, and animal feed.

The term “animal feed” refers to food that is given to animals, such as livestock and pets. Some feeds provide a healthy and nutritious diet, while others may be lacking in nutrients. Animals are given a wide range of different feeds, but the two major types of animal feed are processed animal feeds (compound feed) and fodder.

Compound feeds are feedstuffs that are blended from various raw materials and additives. The main ingredients used in commercially prepared feed are the feed grains, which include corn, soybeans, sorghum, oats, and barley. These blends are formulated according to the specific requirements of the target animal (including different types of livestock and pets).

They are manufactured by feed compounders as meal type, pellets or crumbles.

Compound feeds can be complete feeds that provide all the daily required nutrients, concentrates that provide a part of the ration (protein, energy) or supplements that only provide additional micro-nutrients such as minerals and vitamins.

Oxidation and therefore the shelf life of animal feed ingredients is a common problem in the industry. Oxidation is an irreversible chemical reaction in which oxygen reacts with feed and feed components and can result in decreased animal health and performance. The negative effects of oxidation can be seen in loss of palatability, degradation of the oil component, development of unwanted breakdown products, changes in color, and loss of energy. Meat obtained from animals grown on oxidized feed has significantly lower oxidative status compared to animals fed a feed that has not undergone significant oxidation. Meat from animals fed diets containing high oleic corn products show extended shelf life and greater oxidative stability (PCT Publication WO/2006/002052, published Jan. 5, 2006), particularly when combined with antioxidants such as tocols. Therefore it is highly desirable to prevent oxidation of feed and feed ingredients to protect both nutritional value and organoleptic quality.

Synthetic antioxidants are used to preserve feed quality by preventing the oxidation of lipids, which can lead to improved animal performance. Generally, synthetic antioxidants can act as free radical scavengers and thereby reduce lipid oxidation. Synthetic antioxidants can prolong animal Feed shelf-life and protect nutritional and organoleptic quality

Methods for obtaining soy protein products are well known to those skilled in the art. For example soybean protein products can be obtained in a variety of ways. Conditions typically used to prepare soy protein isolates have been described by (Cho, et al, (1981) U.S. Pat. No. 4,278,597; Goodnight, et al. (1978) U.S. Pat. No. 4,072,670). Soy protein concentrates are produced by three basic processes: acid leaching (at about pH 4.5), extraction with alcohol (about 55-80%), and denaturing the protein with moist heat prior to extraction with water. Conditions typically used to prepare soy protein concentrates have been described by Pass ((1975) U.S. Pat. No. 897,574) and Campbell et al. ((1985) in New Protein Foods, ed. by Altschul and Wilcke, Academic Press, Vol., Chapter 10, Seed Storage Proteins, pp 302-338).

“Soybean-containing products” or “Soy products” can be defined as those products containing/incorporating a soy protein product.

For example, “soy protein products” can include, and are not limited to, those items listed in Table 2.

TABLE 2
Soy Protein Products Derived from Soybean Seeds^{a}

| Whole Soybean Products | Processed Soy Protein Products |
|---|---|
| Roasted Soybeans | Full Fat and Defatted Flours |
| Baked Soybeans | Soy Grits |
| Soy Sprouts | Soy Hypocotyls |
| Soy Milk | Soybean Meal |
| | Soy Milk |
| | Soy Milk Powder |
| | Soy Protein Isolates |
| Specialty Soy Foods/Ingredients | |
| Soy Milk | Soy Protein Concentrates |
| Tofu | Textured Soy Proteins |
| Tempeh | Textured Flours and Concentrates |
| Miso | Textured Concentrates |
| Soy Sauce | Textured Isolates |
| Hydrolyzed Vegetable Protein | Soy Crisps |
| Whipping Protein | |

^{a}See Soy Protein Products: Characteristics, Nutrflonal Aspects and Utilization (1987). Soy Protein Council.

“Processing” refers to any physical and chemical methods used to obtain the products listed in Table 2 and includes, and is not limited to, heat conditioning, flaking and grinding, extrusion, solvent extraction, or aqueous soaking and extraction of whole or partial seeds. Furthermore, “processing” includes the methods used to concentrate and isolate soy protein from whole or partial seeds, as well as the various traditional Oriental methods in preparing fermented soy food products. Trading Standards and Specifications have been established for many of these products (see National Oilseed Processors Association Yearbook and Trading Rules 1991-1992).

Defatted flakes refer to flaked, dehulled cotyledons that have been defatted and treated with controlled heat to remove the remaining hexane. This term can also refer to a flour or grit that has been ground.

“White” flakes refer to flaked, dehulled cotyledons that have been defatted and treated with controlled heat to remove the remaining hexane. This term can also refer to a flour that has been ground.

“Grits” refer to defatted, dehulled cotyledons having a U.S. Standard screen size of between No. 10 and 80.

“Soy Protein Concentrates” refer to those products produced from dehulled, defatted soybeans and typically contain 65 wt % to 90 wt % soy protein on a moisture free basis. Soy protein concentrates are typically manufactured by three basic processes: acid leaching (at about pH 4.5), extraction with alcohol (about 55-80%), and denaturing the protein with moist heat prior to extraction with water. Conditions typically used to prepare soy protein concentrates have been described by Pass (1975) U.S. Pat. No. 3,897,574; Campbell et al., (1985) in New Protein Foods, ed. by Altschul and Wilcke, Academic Press, Vol. 5, Chapter 10, Seed Storage Proteins, pp 302-338).

As used herein, the term “soy protein isolate” or “isolated soy protein” refers to a soy protein containing material that contains at least 90% soy protein by weight on a moisture free basis.

“Extrusion” refers to processes whereby material (grits, flour or concentrate) is passed through a jacketed auger using high pressures and temperatures as a means of altering the texture of the material “Texturing” and “structuring” refer to extrusion processes used to modify the physical characteristics of the material. The characteristics of these processes, including thermoplastic extrusion, have been described previously (Atkinson (1970) U.S. Pat. No. 3,488,770, Horan (1985) In New Protein Foods, ed. by Altschul and Wilcke, Academic Press, Vol. 1A, Chapter 8, pp 367-414). Moreover, conditions used during extrusion processing of complex foodstuff mixtures that include soy protein products have been described previously (Rokey (1983) Feed Manufacturing Technology III, 222-237; McCulloch, U.S. Pat. No. 4,454,804).

Residual fatty acid analysis. The commercial process used to de-fat soy flakes with hexane leaves a residue of fatty acids that can act as substrate for generation of off-flavor compounds. Depending on the method of analysis, the residual fat content of hexane-defatted soy flakes can range from, 0.6-1.0% (W:W) (ether extractable; AOCS Method 920.39 (Official Methods of Analysis of the AOAC International (1995), 16^{th} Edition, Method 920.390, Locator #4.2.01 (modified)) to 2.5-3% (W:W) (acid hydrolysable; AOAC Method 922.06 (Official Methods of Analysis of the AOAC International (1995), 16^{th} Edition, Method 922.06, Locator 32.1.13 (modified)). The principle reason for the discrepancy between these two methods of estimating residual fatty acids is the chemical nature of the fat classes associated with the protein matrix after hexane extraction. A small proportion of the residual fatty acid is in the form of neutral lipid (i.e., triglyceride) and the remainder is present as polar lipid (e.g., phospholipids, a.k.a., lecithin). Because of its polar nature the phospholipid is inaccessible to ether extraction and is only removed from the protein matrix if acid hydrolysis or some other stringent extraction protocol is performed. Therefore, the ether extraction technique gives an estimation of the neutral lipid fraction whereas the acid hydrolysable method gives a better estimate of the total residual fatty acid content (i.e., neutral and polar fractions).

Both of the AOAC methods described above rely on gravimetric determinations of the residual fatty acids and, although in combination they give an indication of the fat classes (neutral vs. polar), such estimates are crude and are subject to interference from other hydrophobic materials (e.g. saponins). Further, no information is obtained on the fatty add composition and how it may have been affected by various experimental treatments or by the genetics of the starting material. AOAC methods for the determination of the fatty acid composition of residual fatty acids are available (Official Methods of Analysis of the AOAC International (2000), 17^{th} Edition, Method 983.23 Locator 45.4.02, Method 969.33 Locator 41.1.28, Method 996.06 Locator 41.1.28A). These are based on the conversion of residual fatty acids, extracted by acid hydrolysis, to fatty acid methyl esters prior to analysis by gas chromatography. Such techniques are rarely used to assess the residual fatty acid content of food materials in commercial settings although they are used for fatty acid evaluations in support of nutritional labeling. A report in which these methods have been used to determine the residual fatty acid composition of commercial soy protein isolates has recently been published (Solina at al. (2005) Volatile aroma components of soy protein isolate and acid-hydrolysed vegetable protein Food Chemistry 90: 861-873)

A facile method for determining the fatty acid composition of the residual fats in soy protein products is described in Example 24. The advantage of this method over others is that it requires no extraction of the residual fats from the matrix prior to derivatization for GC analysis, Further, the technique is suitable for all forms of fatty acids i.e., whether they are initially present as free fatty acids or as fatty acid esters e.g., tri-glycerides or phospholipids (Chistie (1989) Gas Chromatography and Lipids; The Oily Press. Ayr, Scotland). The technique will also remove fatty acids from the protein matrix even if the polar head group of the phospholipid is covalently bound to the protein.

Also, within the scope of this invention are food, food supplements, food bars, and beverages as well as animal feed (such as pet foods) that have incorporated therein a soybean protein product of the invention. The beverage can be in a liquid or in a dry powdered form.

The foods to which the soybean protein product of the invention can be incorporated/added include almost all foods, beverages and feed (such as pet foods). For example, there can be mentioned food supplements, food bars, meats such as meat alternatives, ground meats, emulsified meats, marinated meats, and meats injected with a soybean protein product of the invention. Included may be beverages such as nutritional beverages, sports beverages, protein-fortified beverages, juices, milk, milk alternatives, and weight loss beverages. Mentioned may also be cheeses such as hard and soft cheeses, cream cheese, and cottage cheese. Included may also be frozen desserts such as ice cream, ice milk, low fat frozen desserts, and non-dairy frozen desserts. Finally, yogurts, soups, puddings, bakery products, salad dressings, spreads, and dips (such as mayonnaise and chip dips) may be included.

A soy protein product can be added in an amount selected to deliver a desired amount to a food and/or beverage. The terms “soybean protein product” and “soy protein product” are used interchangeably herein.

Any soybean seed with a combined linoleic and linolenic acid content less than that of commodity soybeans (about 50-60% on a total oil basis), whether it was obtained by transgenic or non-transgenic means, can be used as a blending source of the soy protein product.

Soybeans with decreased levels of saturated fatty acids have been described resulting from mutation breeding (Erickson et al. (1994) J. Hered. 79:465-468; Schnebly at al. (1994) Crop Sci. 34:829-833; and Fehr at al. (1991) Crop Sci. 31:88-89) and transgenic modification (U.S. Pat. No. 5,530,186). Soybeans with decreased levels of polyunsaturated fatty acids have been described resulting from mutation breeding and selection. Reduced levels of linolenic acid have been achieved at relatively constant linoleic acid (U.S. Pat. No. 5,710,369 and U.S. Pat. No. 5,986,118). Decreased linoleic and linolenic acids combined have also been achieved using mutation breeding, genetic crosses and selection (Rahman, S. M. et al. (2001) Crop Sci. 41:26-29). These methods produced soybean seeds with oil profiles having linolenic acid contents of from 1% to 3% of the total fatty acids and total levels of polyunsaturated fatty acids of about 30 to 35% as compared to greater than 6% linolenic acid and greater than 50% total polyunsaturated fatty acids in commodity soybeans.

The discovery of a method for altering the expression of the enzymes responsible for introduction of the second (international patent publication WO 94/11516) and third (international patent publication WO 93/11245) double bonds into soybean seed storage lipid in a directed manner has allowed the production of soybeans with a high mono-unsaturated, very low polyunsaturated fatty acid content and especially a very low linolenic acid content. The genetic combination of these two transgene profiles described in U.S. Pat. No. 6,426,448 leads to a soybean line with minimal poly-unsaturates and high mono-unsaturates and extreme environmental stability of the seed fatty acid profile.

The gene for microsomal delta-12 fatty acid desaturases described in WO 94/11516, can be used to make a high oleic acid soybean variety. The resulting high oleic acid soybean variety was one in which the polyunsaturated fatty acids were reduced from 70% of the total fatty acids to less than 5%.

Two soybean fatty acid desaturases, designated FAD2-1 and FAD2-2, are Δ-12 desaturases that introduce a second double bond into oleic acid to form linoleic acid, a polyunsaturated fatty acid. FAD2-1 is expressed only in the developing seed (Heppard et al. (1996) Plant Physiol. 110:311-319). The expression of this gene increases during the period of oil deposition, starting around 19 days after flowering, and its gene product is responsible for the synthesis of the polyunsaturated fatty acids found in soybean oil. GmFad 2-1 is described in detail by Okuley, J. et al. (1994) Plant Cell 6:147-158 and in WO94/11516. It is available from the ATCC in the form of plasmid pSF2-169K (ATCC accession number 69092). FAD 2-2 is expressed in the seed, leaf, root and stem of the soy plant at a constant level and is the “housekeeping” 12-desaturase gene. The Fad 2-2 gene product is responsible for the synthesis of polyunsaturated fatty acids for cell membranes.

Since FAD2-1 is the major enzyme of this type in soybean seeds, reduction in the expression of FAD2-1 results in increased accumulation of oleic acid (18:1) and a corresponding decrease in polyunsaturated fatty acid content.

Reduction of expression of FAD2-2 in combination with FAD2-1 leads to a greater accumulation of oleic acid and corresponding decrease in polyunsaturated fatty acid content.

FAD3 is a Δ-15 desaturase that introduces a third double bond into linoleic acid (18:2) to form linolenic acid (18:3). Reduction of expression of FAD3 in combination with reduction of FAD2-1 and FAD2-2 leads to a greater accumulation of oleic acid and corresponding decrease in polyunsaturated fatty add content, especially linolenic add.

Nucleic add fragments encoding FAD2-1, FAD2-2, and FAD3 have been described in WO 94/11516 and WO 93/11245. Chimeric recombinant constructs comprising all or a part of these nucleic add fragments or the reverse complements thereof operably linked to at least one suitable regulatory sequence can be constructed wherein expression of the chimeric gene results in an altered fatty acid phenotype. A chimeric recombinant construct can be introduced into soybean plants via transformation techniques well known to those skilled in the art.

Transgenic soybean plants resulting from a transformation with a recombinant DNA are assayed to select plants with altered fatty acid profiles. The recombinant construct may contain all or part of 1) the FAD2-1 gene or 2) the FAD2-2 gene or 3) the FAD3 gene or 4) combinations of all or portions of the FAD2-1, Fad2-2, or FAD3 genes.

Recombinant constructs comprising all or part of 1) the FAD2-1 gene with or without 2) all or part of the Fad2-2 gene with or without all or part of the FAD3 gene can be used in making a transgenic soybean plant having a high oleic phenotype. An altered fatty acid profile, specifically an increase in the proportion of oleic acid and a decrease in the proportion of the polyunsaturated fatty acids, indicates that one or more of the soybean seed FAD genes (FAD2-1, Fad2-2, FAD3) have been suppressed. Assays may be conducted on soybean somatic embryo cultures and seeds to determine suppression of FAD2-1, Fad2-2, or FAD3.

It is well understood by those skilled in the art that recombinant constructs comprising sequences other than those specifically exemplified which have similar functions, may be used. These constructs may include any seed-specific promoter. These constructs may or may not also include any nucleotides that promote stem-loop formation. These constructs may contain a polynucleotide having a nucleotide sequence identical to any portion of the gene or genes mentioned above inserted in sense or anti-sense orientation with respect to the promoter. Finally, these constructs may or may not contain any transcription termination signal.

It was observed that when the commodity and high oleic flake slurries were mixed together and processed further to the neutralized curd slurry, the slurry viscosity was much lower. With as little as 40% high oleic flake, there was a large drop in viscosity of the resultant in-process slurry. This allows the functionality of high oleic isolate to be obtained by blending only a small amount of it with the commodity isolate. It also shows processing benefits like improvement in the solids to dryer due to drastically reduced viscosity of the mixture. One could also gain similar viscosity manipulation benefits by modulating the fatty acid profiles by genetic manipulation or by selective breeding, e.g. mid oleic beans. The resultant isolates may be used for various applications ranging from milk like products e.g. soy milk with viscosities in range of 1-10 GP to puddings or applications like gels and meat analogues.

Surprisingly, it has been found that addition of a small amount of polyunsaturated free fatty acids, such as a mixture of linoleic and linolenic acids, to defatted high oleic flake at the time of extraction of the protein leads to a dramatic increase in the viscosity of the extracted soy protein. The combination of appropriate levels of PUFAs and high oleic flake can therefore be used to generate soy proteins having any desired viscosity between the low value of isolate from high oleic flake and the high value of isolate obtained from commodity flake. These isolated soy proteins with different viscosities are desirable for different food applications, for example most beverages require lower viscosity while some beverages and solid foods require higher viscosity.

The viscosity of the in-process protein was found to increase incrementally in a non-linear fashion with increasing amounts of added PUFA mixture. The slope of a plot of the viscosity vs amount of added PUFA decreases with increasing PUFA. At higher levels of PUFA addition the viscosity of the in-process protein isolated from high oleic flake exceeded that isolated from the commodity flake.

The extent of the viscosity increase in the isolated protein is dependent on the degree of unsaturation of the added fatty acid. The viscosity of the in-process protein generally increased as the number of double bonds in the fatty acid increased.

The increase in viscosity with the addition of PUFAs was accompanied by a decrease in Whiteness index.

### EXAMPLES

The present invention is further defined in the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

The production of high oleic soybean lines is described in detail in Examples 3, 5 and 8, but is not limited to the methods described therein.

### Example 1

### Transformation of Soybean (Glycine max)

### Embryo Cultures and Regeneration of Soybean Plants.

Soybean embryogenic suspension cultures are transformed by the method of particle gun bombardment using procedures known in the art (Klein et al. (1987) Nature (London) 327:70-73; U.S. Pat. No. 4,945,050; Hazel et al. (1998) Plant Cell. Rep. 17:765-772; Samoylov et al. (1998) In Vitro Cell Dev. Biol.-Plant 34:8-13). In particle gun bombardment procedures it is possible to use purified 1) entire plasmid DNA or, 2) DNA fragments containing only the recombinant DNA expression cassette(s) of interest.

Stock tissue for transformation experiments are obtained by initiation from soybean immature seeds. Secondary embryos are excised from explants after 6 to 8 weeks on culture initiation medium. The initiation medium is an agar-solidified modified MS (Murashige and Skoog (1962) Physiol. Plant. 15:473-497) medium supplemented with vitamins, 2,4-D and glucose. Secondary embryos are placed in flasks in liquid culture maintenance medium and maintained for 7-9 days on a gyratory shaker at 26-1+/−2° C. under ˜80 μEm-2 s-1 light intensity. The culture maintenance medium is a modified MS medium supplemented with vitamins, 2,4-D, sucrose and asparagine. Prior to bombardment, clumps of tissue are removed from the flasks and moved to an empty 60×15 mm petri dish for bombardment. Tissue is dried by blotting on Whatman #2 filter paper. Approximately 100-200 mg of tissue corresponding to 10-20 clumps (1-5 mm in size each) are used per plate of bombarded tissue.

After bombardment, tissue from each bombarded plate is divided and placed into two flasks of liquid culture maintenance medium per plate of bombarded tissue. Seven days post bombardment, the liquid medium in each flask is replaced with fresh culture maintenance medium supplemented with 100 ng/ml selective agent (selection medium). For selection of transformed soybean cells the selective agent used can be a sulfonylurea (SU) compound with the chemical name, 2-chloro-N-((4-methoxy-6 methyl-1,3,5-triazine-2-yl)aminocarbonyl)benzenesulfonamide (common names: DPX-W4189 and chlorsulfuron). Chlorsulfuron is the active ingredient in the DuPont sulfonylurea herbicide, GLEAN®. The selection medium containing SU is replaced every week for 6-8 weeks. After the 6-8 week selection period, islands of green, transformed tissue are observed growing from untransformed, necrotic embryogenic clusters. These putative transgenic events are isolated and kept in media with SU at 100 ng/ml for another 2-6 weeks with media changes every 1-2 weeks to generate new, clonally propagated, transformed embryogenic suspension cultures. Embryos spend a total of around 8-12 weeks in contact with SU. Suspension cultures are subcultured and maintained as clusters of immature embryos and also regenerated into whole plants by maturation and germination of individual somatic embryos.

### Example 2

### Fatty Acid Analysis of Soybeans

In order to determine altered fatty acid composition as a result of suppression of the fatty acid desaturase, the relative amounts of the fatty acids, palmitic, stearic, oleic, linoleic and linolenic, can be determined as follows. Fatty acid methyl esters are prepared from single, mature, somatic soybean embryos or soybean seed chips by transesterification. One embryo, or a chip from a seed, is placed in a vial containing 50 μL of trimethylsulfonium hydroxide and incubated for 30 minutes at room temperature while shaking. After 30 minutes 0.5 mL of hexane is added, the sample is mixed and allowed to settle for 15 to 30 minutes to allow the fatty acids to partition into the hexane phase. Fatty acid methyl esters (5 μL from hexane layer) are injected, separated, and quantified using a Hewlett-Packard 6890 Gas Chromatograph fitted with an Omegawax 320 fused silica capillary column (Supelco Inc., Cat#24152). The oven temperature is programmed to hold at 220° C. for 2.7 minutes, increase to 240° C. at 20° C. per minute, and then hold for an additional 2.3 minutes. Carrier gas is supplied with a Whatman hydrogen generator. Retention times were compared to those for methyl esters of commercially available standards (Nu-Chek Prep, Inc. catalog #U-99-A).

### Example 3

### Production of Soybeans with High Levels Oleic Acid and/or High Levels of Stearic Acid and/or Low Levels of Polyunsaturated Fatty Acids by Suppression of Fatty Acid Desaturases

Recombinant DNA fragments were prepared and used in transformation of soybean for the simultaneous suppression of fatty acid desaturases FAD2-1 and FAD2-2 and fatty acid desaturase FAD3. A description of the construction of the recombinant DNA fragments follows.

### A. Recombinant DNA Fragment PHP21676A

Recombinant DNA fragment PHP21676A contains a gene expression silencing cassette designed to silence expression of the FAD2-1 and FAD2-2 genes, and the FAD3 gene, linked in a head to head configuration to the ALS selectable marker recombinant DNA fragment of Example 1D below. The nucleotide sequence of recombinant DNA fragment PHP21676A is shown in SEQ ID NO:1, Recombinant DNA fragment PHP21676A contains in 5′ to 3′ orientation;
a) the complementary strand of the ALS selectable marker recombinant DNA fragment of Example 10 below,
b) about 2088 nucleotides of the Kti3 promoter,
c) a 74-nucleotide synthetic sequence,
d) an approximately 1500 polynucleotide fragment comprising about 470 nucleotides from the soybean FAD2-2 gene, 420 nucleotides from the soybean FAD2-1 gene, and 643 nucleotides from the soybean FAD3 gene inserted at a unique Not I restriction endonuclease site,
e) an inverted repeat of the 74-nucleotide synthetic sequence in c), and
f) about 202 nucleotides of the Kti3 transcription terminator.

The sequence of the approximately 1500 polynucleotide fragment of item d) above is shown in SEQ ID NO:2. The approximately 1500 polynucleotide fragment comprising about 470 nucleotides from the soybean FAD2-2 gene, about 420 nucleotides from the soybean FAD2-1 gene, about 643 nucleotides from the soybean FAD3 gene was constructed by PCR amplification as follows.

An approximately 0.9 kb DNA fragment, comprising a portion of the soybean FAD2-2 gene and a portion of the soybean FAD2-1 gene, was obtained by PCR amplification using primers BM35 (SEQ ID NO:3) and BM39 (SEQ ID NO:4) and using as a template, recombinant DNA fragment KSFAD2-hybrid, described in Example 1B below.

An approximately 0.65 kb DNA fragment, comprising a portion of a FAD3 gene, was obtained by PCR amplification using primers BM40 (SEQ ID NO:5) and BM41 (SEQ ID NO:6) and using plasmid pXF1 as template. Plasmid pXF1 comprises a polynucleotide encoding a soybean delta-15 desaturase (FAD3) and is described in U.S. Pat. No. 5,952,544 issued on Sep. 14, 1999. Plasmid pXF1 was deposited with the American Type Culture Collection (ATCC) of Rockville, Md. on Dec. 3, 1991 under the provisions of the Budapest Treaty, and bears Accession Number ATCC 68874.

The approximately 0.9 kb fragment, comprising a portion of the soybean FAD2-2 gene and a portion of the soybean FAD2-1 gene, and the approximately 0.65 kb fragment, comprising a portion of a FAD3 gene, were mixed and used as template for a PCR amplification with BM35 and BM41 as primers to yield an approximately 1533 bp fragment that was cloned into the commercially available plasmid pCR2.1 using the TOPO TA Cloning Kit (Invitrogen).

After digestion with NotI the approximately 1500 bp fragment having the nucleotide sequence shown in SEQ ID NO:2 was ligated into the NotI site of plasmid pKS210 (Example 1C below).
B. Recombinant DNA Fragment KSFAD2-hybrid

Recombinant DNA Fragment KSFAD2-hybrid contains an approximately 890 polynucleotide fragment comprising about 470 nucleotides from the soybean FAD2-2 gene and 420 nucleotides from the soybean FAD2-1 gene. The nucleotide sequence of recombinant DNA fragment KSFAD2-hybrid is shown in SEQ ID NO:7 Recombinant DNA Fragment KSFAD2-hybrid was constructed as follows.

An approximately 0.47 kb DNA fragment comprising a portion of the soybean FAD2-2 gene was obtained by PCR amplification using primers KS1 (SEQ ID NO:8) and KS2 (SEQ ID NO:9) and using genomic DNA purified from leaves of Glycine max cv. Jack as a template.

An approximately 0.42 kb DNA fragment comprising a portion of the soybean FAD2-1 gene was obtained by PCR amplification using primers KS3 (SEQ ID NO:10) and KS4 (SEQ ID NO:11) and using genomic DNA purified from leaves of Glycine max cv. Jack as a template.

The 0.47 kb fragment comprising a portion of the soybean FAD2-2 gene and the 0.42 kb fragment comprising a portion of the soybean FAD2-1 gene were gel purified using GeneClean (Qbiogene, Irvine, Calif.), mixed, and used as template for PCR amplification with KS1 and KS4 as primers to yield an approximately 890 bp fragment that was cloned into the commercially available plasmid pGEM-T Easy (Promega, Madison, Wis.) to create a plasmid comprising recombinant DNA Fragment KSFAD2-hybrid.

C. Preparation of Plasmid pKS210 and Plasmid PHP17731

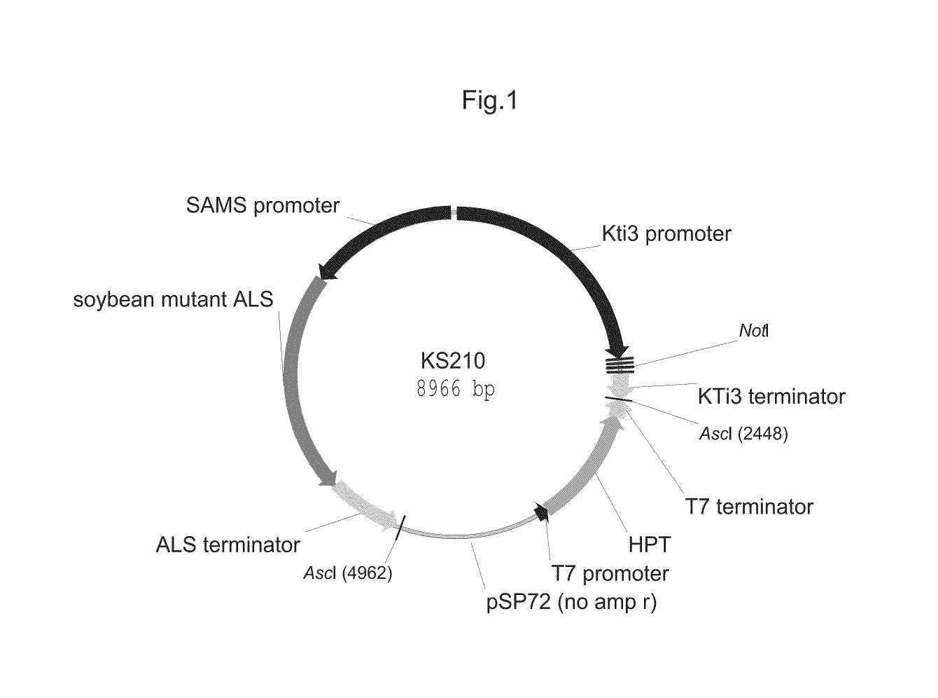

Plasmid pKS210 is derived from the commercially available cloning vector pSP72 (Promega). The beta lactamase coding region has been replaced by a hygromycin phosphotransferase gene for use as a selectable marker in E. coli. In addition, a gene expression silencing cassette linked in a head to head configuration to the ALS selectable marker recombinant DNA fragment of Example 1B has been added. The gene expression silencing cassette in plasmid pKS210 comprises the KTi3 promoter, a 74 nucleotide synthetic sequence, a unique NotI restriction endonuclease site, an inverted repeat of the 74 nucleotide synthetic sequence, and the Kti3 terminator region. The gene encoding Kti3 has been described (Jofuku and Goldberg (1989) Plant Cell 1:1079-1093). The 74-nucleotide synthetic sequences of c) and e) (above) promote formation of a stem structure. Insertion of a nucleotide fragment from a desired gene in the unique Not I site has been shown to result in suppression of the desired gene as described in PCT Publication WO 02/00904, published 3 Jan. 2002. The nucleotide sequence of this seed-specific gene expression-silencing cassette from pKS133 is shown in SEQ ID NO:12. A map of plasmid pKS210 is shown in FIG. 1 and its nucleotide sequence is disclosed in SEQ ID NO:13.

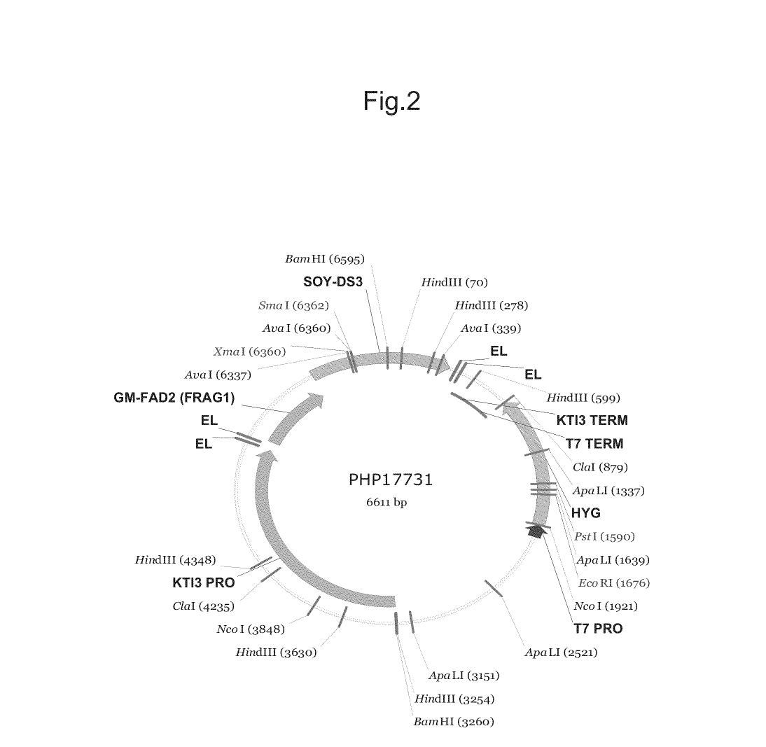

The recombinant plasmid PHP17731, containing gene sequences for the simultaneous silencing of one of the soybean delta-9 desaturase genes and the soybean delta-12 desaturase gene FAD2-1, was prepared. The soybean KTI promoter, terminator regions along with a synthetic inverted repeat sequence were taken from plasmid KS133 (WO 2002016565A2, A3). A fragment of the FAD2-1 gene was amplified by PCR using soybean genomic DNA and the sequence in SEQ ID NO 5 of U.S. Pat. No. 6,372,965 B1 as template to produce the fragment of base pairs 5423 to 6033 of SEQ ID NO:14 (PHP17731), Adjacent to that fragment a portion of the coding sequence of copy 3 of the soybean delta-9 desaturase (sequence 1 of WO 2002016565A2, A3) was placed, which now comprises bases 6054 to 411 of PHP17731, Fragment PHP17731A (SEQ ID NO:15) was removed from cloning vector PHP17731 by digestion with restriction endonuclease AscI and purified as described in section E below. A map of plasmid PHP17731 is shown in FIG. 2.

### D. ALS Selectable Marker Recombinant DNA Fragment

A recombinant DNA fragment comprising a constitutive promoter directing expression of a mutant soybean acetolactate synthase (ALS) gene followed by the soybean ALS 3′ transcription terminator was used as a selectable marker for soybean transformation. The constitutive promoter used is a 1.3-Kb DNA fragment that functions as the promoter for a soybean S-adenosylmethionine synthase (SAMS) gene and is described in POT publication No. WO 00137662 published 29 Jun. 2000. The nucleotide sequence of this recombinant DNA fragment used as a selectable marker is shown in SEQ ID NO:16. The mutant soybean ALS gene encodes an enzyme that is resistant to inhibitors of ALS, such as sulfonylurea herbicides. The deduced amino acid sequence of the mutant soybean ALS present in the recombinant DNA fragment used as a selectable marker is shown in SEQ ID NO:17.

Mutant plant ALS genes encoding enzymes resistant to sulfonylurea herbicides are described in U.S. Pat. No. 5,013,659. One such mutant is the tobacco SURB-Hra gene, which encodes a herbicide-resistant ALS with two substitutions in the amino acid sequence of the protein. This tobacco herbicide-resistant ALS contains alanine instead of proline at position 191 in the conserved “subsequence B” (SEQ ID NO:18) and leucine instead of tryptophan at position 568 in the conserved “subsequence F” (SEQ ID NO:19) (U.S. Pat. No. 5,013,659; Lee et al. (1988) EMBO J. 7:1241-1248).

The ALS selectable marker recombinant DNA fragment was constructed using a polynucleotide for a soybean ALS to which the two Hra-like mutations were introduced by site directed mutagenesis. Thus, this recombinant DNA fragment will translate to a soybean ALS having alanine instead of praline at position 183 and leucine instead of tryptophan at position 560.

In addition, during construction of the SAMS promoter-mutant ALS expression cassette, the coding region of the soybean ALS gene was extended at the 5′-end by five additional codons, resulting in five amino acids (M-R-H-N-T; SEQ ID NO:20), added to the amino-terminus of the ALS protein. These extra amino acids are adjacent to and presumably removed with the transit peptide during targeting of the mutant soybean ALS protein to the plastid. A DNA fragment comprising a polynucleotide encoding the soybean ALS was digested with KpnI, blunt ended with T4 DNA polymerase, digested with SalI, and inserted into a plasmid containing the SAMS promoter which had been previously digested with NcoI and blunt ended by filling-in with Klenow DNA polymerase.

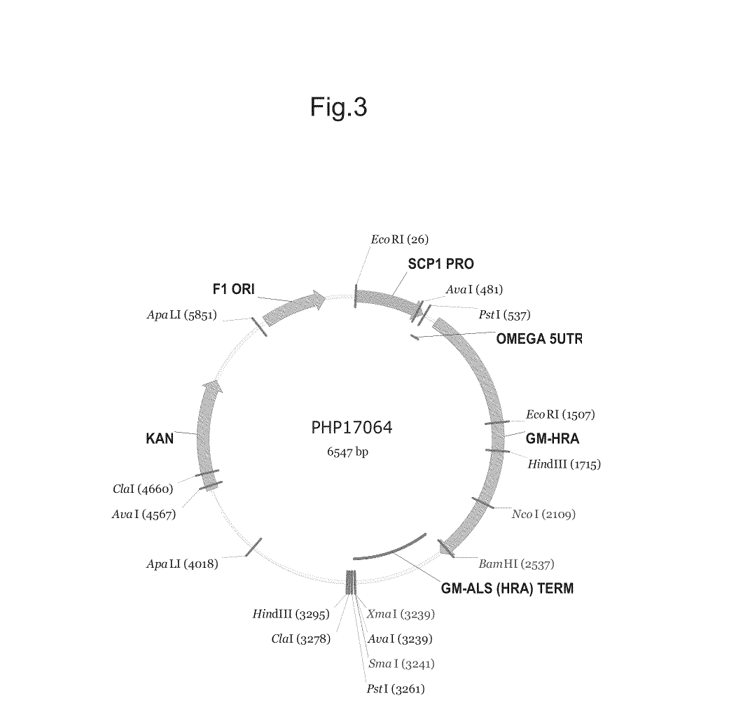

A second selectable marker plasmid and subsequent fragment was prepared by substituting an alternative constitutively expressed plant promoter for the SAMS promoter described above. The synthetic promoter SCP1 (U.S. Pat. No. 6,072,050) was placed in front of the mutant soybean ALS coding sequence to form plasmid PHP17064 (SEQ ID NO: 21 and FIG. 3). For use in soybean transformation fragment PHP17064A (SEQ ID NO:22) was excised from its cloning vector using restriction endonuclease XbaI and purified as described in section E below.

E. Preparation of Recombinant DNA Fragments. PHP21676A, PHP17731A and PHP17064A, for Soybean Transformation.

For use in plant transformation experiments, the 7993 bp recombinant DNA fragment PHP21676A was removed from its cloning plasmid using restriction endonuclease AscI. Each one of the recombinant DNA fragments PHP21676A, PHP17731A and PHP17064A was separated from the remaining plasmid DNA by agarose gel electrophoresis. Precipitation of the recombinant DNA fragment onto gold particles and soybean transformation was performed as described in Example 1. For every eight bombardment transformations, 30 μl of solution were prepared with 3 mg of 0.6 μm gold particles and 1 to 90 picograms (pg) of DNA fragment per base pair of DNA fragment.

Alternatively, mixtures of fragments PHP17064A and PHP17731A at either equal parts or two parts PHP17731A to PHP17064A were added to gold particles at the same weight per base pair as described above and used in transformation to silence the delta-9 and delta-12 desaturase genes.

### Example 4

### Fatty acid analysis of Soybean Transformed with Recombinant DNA Fragments PHP21676A and with PHP17064A and PHP17731A Combined

In a soybean transformation experiment using recombinant DNA fragment PHP21676A as described above, 67 independently transformed embryogenic suspension cultures found to be resistant to sulfonylurea herbicide were obtained. An increase in oleic acid as a percentage of the five major fatty acids, palmitic, stearic, oleic, linoleic and linolenic, is indicative of suppression of the FAD2 genes. Thirteen of the 67 herbicide resistant embryogenic suspension cultures (19%) produced somatic embryos with greater than 25% oleic acid, compared to about 8% oleic acid for untransformed embryos.

Plants were regenerated and T1 seeds were produced from 9 of the 13 events. Seeds were tested for suppression of fatty acid desaturases by measuring fatty acid composition of the seed oil as described in Example 2. Plants derived from 5 transformation events produced seeds exhibiting the high oleic acid-low polyunsaturated fatty acid phenotype.

In a soybean transformation experiment, using the mixture of recombinant DNA fragments PHP17064A and PHP17731A, transformed embryogenic suspension cultures found to be resistant to sulfonylurea herbicide were obtained, screened for the number of copies of the transgene fragments present by southern analysis and then by fatty acid profile of the somatic embryo. A rise in the level of stearic and of oleic acid was taken as indicator of silencing of the seed expressed delta-9 desaturase and delta-12 desaturase. Thirty-three transformed candidate lines were regenerated to mature soybean plants and seed from the initial transformants was analyzed for fatty acid profile. From these lines further selections were made from seed obtained from selfed plants in two additional generations. One candidate line was chosen in which the sum of linoleic and linolenic acid was less than 14% of total fatty acids and in which the stearic acid content was greater than 16% of total fatty acids.

### Example 5

### Genetic Material Used to Produce the High Oleic Trait (Version 1)

High oleic soybeans were prepared by recombinant manipulation of the activity of oleoyl 12-desaturase.

GmFad 2-1 was placed under the control of a strong, seed-specific promoter derived from the α′-subunit of the soybean (Glycine max) β-conglycinin gene. This promoter allows high level, seed specific expression of the trait gene. It spans the 606 bp upstream of the start codon of the α′ subunit of the Glycine max β-congylcinin storage protein. The β-conglycinin promoter sequence represents an allele of the published β-conglycinin gene (Doyle et al., (1986) J. Biol. Chem. 261:9228-9238) having differences at 27 nucleotide positions. It has been shown to maintain seed specific expression patterns in transgenic plants (Barker et al., (1988) Proc. Natl. Acad. Sci. 85:458-462 and Beachy at al., (1985) EMBO J. 4:3047-3053). The reading frame was terminated with a 3′ fragment from the phaseolin gene of green bean (Phaseolus vulgaris). This is a 1174 bp stretch of sequences 3′ of the Phaseolus vulgaris phaseolin gene stop codon (originated from done described in Doyle et al., 1986).

The GmFad 2-1 open reading frame (ORF) was in a sense orientation with respect to the promoter so as to produce a gene silencing of the sense GmFad 2-1 cDNA and the endogenous GmFad 2-1 gene. This phenomenon, known as “sense suppression” is an effective method for deliberately turning off genes in plants and is described in U.S. Pat. No. 5,034,323.

For maintenance and replication of the plasmid in E. coli the GmFad 2-1 transcriptional unit described above was cloned into plasmid pGEM-9z(−) (Promega Biotech, Madison Wis., USA).

For identification of transformed soybean plants the β-glucuronidase gene (GUS) from E. coli was used. The cassette used consisted of the three modules; the Cauliflower Mosaic Virus 35S promoter, the β-glucuronidase gene (GUS) from E. coli and a 0.77 kb DNA fragment containing the gene terminator from the nopaline synthase (NOS) gene of the Ti-plasmid of Agrobacterium tumefaciens. The 353 promoter is a 1.4 kb promoter region from CaMV for constitutive gene expression in most plant tissues (Odell et al. (1985) Nature 303:810-812), the GUS gene a 1.85 kb fragment encoding the enzyme β-glucuronidase (Jefferson et al. (1986) PNAS USA 83:8447-8451) and the NOS terminator a portion of the 3° end of the nopaline synthase coding region (Fraley et al., (1983) PNAS US 80:4803-4807). The GUS cassette was cloned into the GmFad 2-1/pGEM-9z(−) construct and was designated pBS43.

Plasmid pBS43 was transformed into meristems of the elite soybean line A2396, by the method of particle bombardment as described in Example 1. Fertile plants were regenerated using methods well known in the art.

From the initial population of transformed plants, a plant was selected which was expressing GUS activity and which was also positive for the GmFad 2-1 gene (Event 260-05) when evaluated by PCR. Small chips were taken from a number of R1 seeds of plant 260-05 and screened for fatty acid composition. The chipped seed was then planted and germinated. Genomic DNA was extracted from the leaves of the resulting plants and cut with the restriction enzyme Barn H. The blots were probed with a phaseolin probe.

From the DNA hybridization pattern it was clear that in the original transformation event the GmFad 2-1 construct had become integrated at two different loci in the soybean genome. At one locus (Locus A) the GmFad 2-1 construct was causing a silencing of the endogenous GmFad 2-1 gene, resulting in oleic acid contents as shown in Table 3. For comparison elite soybean varieties have an oleic acid content of about 20%. At locus A there were two copies of pBS43. On the DNA hybridization blot this was seen as two cosegregating bands. At the other integration locus (Locus B) the GmFad 2-1 was over-expressing, thus decreasing the oleic acid content to about 4%.

Fourth generation segregant lines (R4 plants), generated from the original transformant, were allowed to grow to maturity. R4 seeds, which contained only the silencing Locus A (e.g., G94-1) did not contain any detectable GmFad 2-1 mRNA (when measured by Northern blotting) in samples recovered 20 days after flowering. GmFad 2-2 mRNA, although reduced somewhat compared with controls, was not suppressed. Thus the GmFad 2-1 sense construct had the desired effect of preventing the expression of the GmFad 2-1 gene and thus increasing the oleic acid content of the seed. All plants homozygous for the GmFad 2-1 silencing locus had an identical Southern blot profile over a number of generations. This indicates that the insert was stable and at the same position in the genome over at least four generations.

### Example 6

### Fatty Acid Analysis High Oleic Trait (Version 1)

A summary of the oleic acid contents found in the different generations of recombinant soybean plants and seeds is presented in Table 7. The Fatty Acid composition was determined as described in Example 2.

TABLE 3

| Plant ID | Generation | Seed | |
|---|---|---|---|
| | Planted^{a} | Analyzed^{a} | Bulk Oleic Acid (%) |
| G253 | R0:1 | R1:2 | 84.1% |
| G276 | R0:1 | R1:2 | 84.2% |
| G296 | R0:1 | R1:2 | 84.1% |
| G313 | R0:1 | R1:2 | 83.8% |
| G328 | R0:1 | R1:2 | 84.0% |
| G168-187 | R1:2 | R2:3 | 84.4% |
| G168-171 | R1:2 | R2:3 | 85.2% |
| G168-59-4 | R2:3 | R3:4 | 84.0% |
| G168-72-1 | R2:3 | R3:4 | 84.1% |
| G168-72-2 | R2:3 | R3:4 | 84.5% |
| G168-72-3 | R2:3 | R3:4 | 84.3% |
| G168-72-4 | R2:3 | R3:4 | 83.3% |

^{a}R0:1 indicates the seed and the plant grown from seed after selfing of the first generation transformant.
R1:2 indicates the seed and the plant grown from seed after selfing of the second generation transformant.
R2:3 indicates the seed and the plant grown from seed after selfing of the third generation transformant.
R3:4 indicates the seed and the plant grown from seed after selfing of the fourth generation transformant.

### Example 7

### Genetic Material Used to Produce the High Oleic Trait (Version 2)

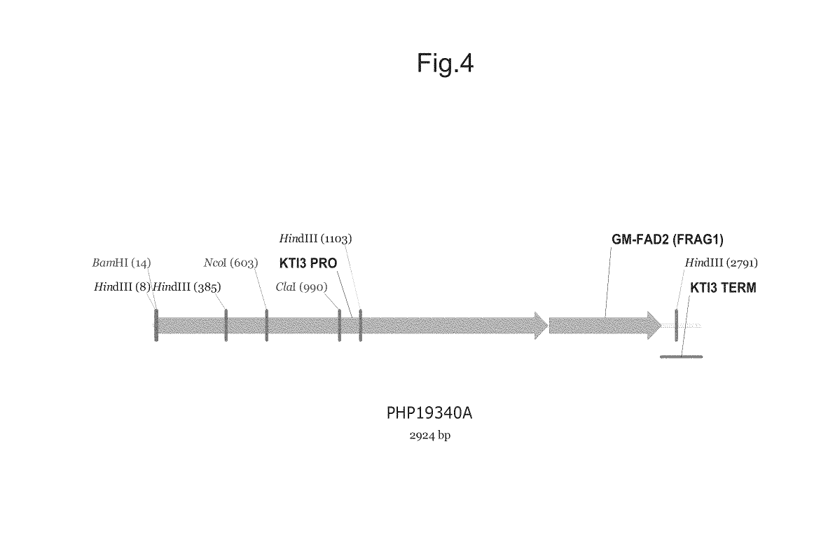

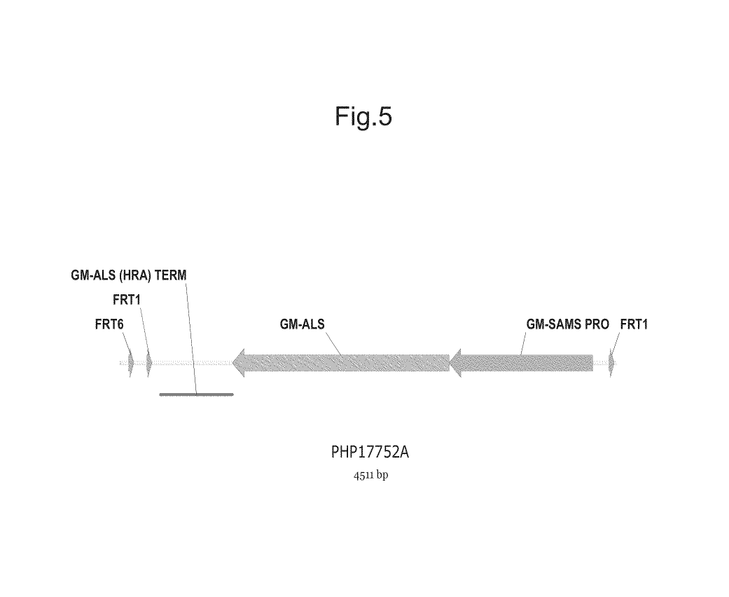

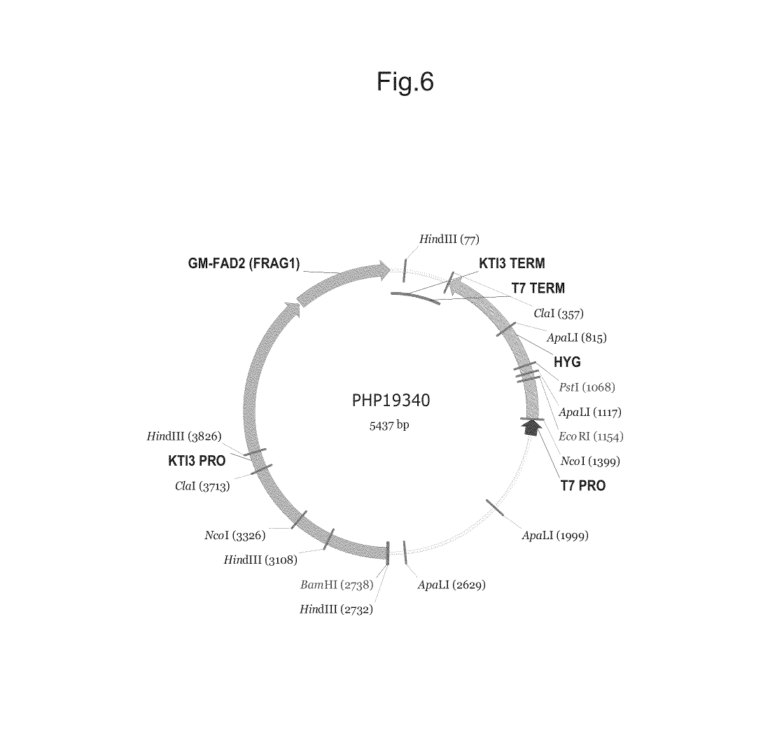

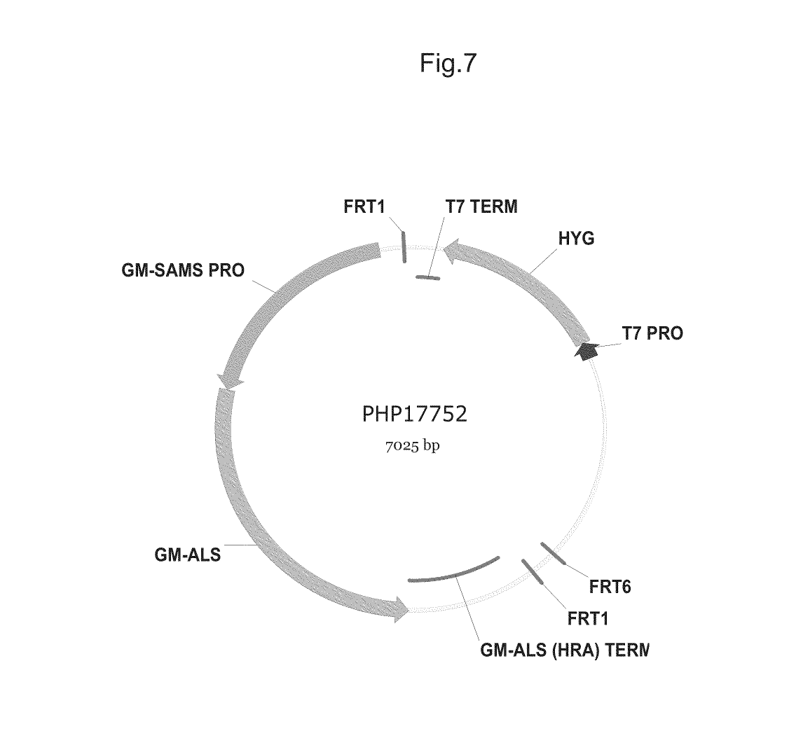

A Soybean (Glycine max) event was produced by particle co-bombardment as described in Example 1 with fragments PHP19340A (FIG. 4; SEQ ID NO:23) and PHP17752A (FIG. 5; SEQ ID NO:24). These fragments were obtained by AscI digestion from a source plasmid. Fragment PHP19340A was obtained from plasmid PHP19340 (FIG. 6; SEQ ID NO:25) and fragment PHP17752A was obtained from plasmid PHP17752 (FIG. 7; SEQ ID NO:26). The PHP19340A fragment contains a cassette with a 597 bp fragment of the soybean microsomal omega-6 desaturase gene 1 (gm-fad2-1) (Heppard et al., 1996, Plant Physiol. 110: 311-319).

The presence of the gm-fad2-1 fragment in the expression cassette acts to suppress expression of the endogenous omega-6 desaturases, resulting in an increased level of oleic acid and decreased levels of palmitic, linoleic, and linolenic acid levels. Upstream of the gm-fad2-1 fragment is the promoter region from the Kunitz trypsin inhibitor gene 3 (KTi3) (Jofuku and Goldberg, 1989, Plant Cell 1: 1079-1093; Jofuku et al, 1989, Plant Cell 1: 427-435) regulating expression of the transcript. The KTi3 promoter is highly active in soy embryos and 1000-fold less active in leaf tissue (Jofuku and Goldberg, 1989, Plant Cell 1: 1079-1093). The 3′ untranslated region of the KTi3 gene (KTi3 terminator) (Jofuku and Goldberg, 1989, Plant Cell 1: 1079-1093) terminates expression from this cassette.

The PHP17752A fragment contains a cassette with a modified version of the soybean acetolactate synthase gene (gm-hra) encoding the GM-HRA protein with two amino acid residues modified from the endogenous enzyme and five additional amino acids at the N-terminal region of the protein derived from the translation of the soybean acetolactate synthase gene 5 untranslated region (Falco and Li, 2003, US Patent Application: 2003/0226166). The gm-hra gene encodes a form of acetolactate synthase, which is tolerant to the sulfonylurea class of herbicides. The GM-HRA protein is comprised of 656 amino acids and has a molecular weight of approximately 71 kDa.

The expression of the gm-lire gene is controlled by the 5′ promoter region of the S-adenosyl-L-methionine synthetase (SAMS) gene from soybean (Falco and Li, 2003, US Patent Application: 2003/0226166). This 5′ region consists of a constitutive promoter and an intron that interrupts the SAMS 5′ untranslated region (Falco and Li, 2003). The terminator for the gm-hra gene is the endogenous soybean acetolactate synthase terminator (als terminator) (Falco and Li, 2003, US Patent Application; 2003/0226166).

### Example 8

### Transformation and Selection for the Soybean High Oleic Event (Version 2)

For transformation of soybean tissue, a linear portion of DNA, containing the gm-fad2-1 gene sequence and the regulatory components necessary for expression, was excised from the plasmid PHP19340 through the use of the restriction enzyme AscI and purified using agarose gel electrophoresis. A linear portion of DNA, containing the gm-hra gene sequences and the regulatory components necessary for expression, was excised from the plasmid PHP17752 through the use of the restriction enzyme AscI and purified using agarose gel electrophoresis. The linear portion of DNA containing the gm-fad2-1 gene is designated insert PHP19340A and is 2924 bp in size. The linear portion of DNA containing the gm-hra gene is designated insert PHP17752A and is 4511 bp in size. The only DNA introduced into transformation event DP-305423-1 was the DNA of the inserts described above.

The transgenic plants from event DP-305423-1 were obtained by microprojectile bombardment as described in Example 1. Embryogenic tissue samples were taken for molecular analysis to confirm the presence of the gm-fad2-1 and gm-hra transgenes by Southern analysis. Plants were regenerated from tissue derived from each unique event and transferred to the greenhouse for seed production.

### Example 9

### Southern Analysis of Plants Containing the High Oleic Event Version 2

Materials and Methods:

Genomic DNA was extracted from frozen soybean leaf tissue of individual plants of the T4 and T5 generations of DR-305423-1 and of control (variety: Jack) using a standard Urea Extraction Buffer method. Genomic DNA was quantified on a spectrofluorometer using Pico Green® reagent (Molecular Probes, Invitrogen). Approximately 4 μg of DNA per sample was digested with Hind III or Nco I. For positive control samples, approximately 3 pg (2 genome copy equivalents) of plasmid PHP19340 or PHP17752 was added to control soybean genomic DNA prior to digestion. Negative control samples consisted of unmodified soybean genomic DNA (variety: Jack). DNA fragments were separated by size using agarose gel electrophoresis.

Following agarose gel electrophoresis, the separated DNA fragments were depurinated, denatured, neutralized in situ, and transferred to a nylon membrane in 20×SSC buffer using the method as described for TURBOBLOTTER™ Rapid Downward Transfer System (Schleicher & Schuell). Following transfer to the membrane, the DNA was bound to the membrane by UV crosslinking.

DNA probes for gm-fad2-1 and gm-hra were labeled with digoxigenin (DIG) by PCR using the PCR DIG Probe Synthesis Kit (Roche).

Labeled probes were hybridized to the target DNA on the nylon membranes for detection of the specific fragments using DIG Easy Hyb solution (Roche) essentially as described by manufacturer. Post-hybridization washes were carried out at high stringency. DIG-labeled probes hybridized to the bound fragments were detected using the CDP-Star Chemiluminescent Nucleic Acid Detection System (Roche). Blots were exposed to X-ray film at room temperature for one or more time points to detect hybridizing fragments. The fatty Acid composition of the event was determined as described in Example 2. Oleic acid levels determined in 29 different events (T1 generation) ranged from 61.5-84.6%. Oleic acid level from one event (T4-T5 generation) ranged from 72-82%.

### Example 10

### Viscosity Measurements of Soy Protein Solutions Prepared at Bench Scale from Defatted Rakes

Rheological measurements were performed in duplicate on a combination of Anton Pear MCR-300 and MCR-301 rheometers. Each rheometer was equipped with a concentric cylinder geometry (Anton Pear 0027) having an active length of 119.2 mm, a position length of 72.5 mm, and a gap length of 40 mm. Temperature control was achieved by circulating 22° C. water from controlled-temperature baths to a Peltier sample heater that controls the temperature of the measuring cell. All measurements were carried out at 25±0.05° C. Viscosity curves for each sample were obtained via the following 4-step procedure: (1) A 19 mL sample was loaded into the concentric cylinder cup and pre-sheared for 30 s at a shear rate of 10 s⁻¹ to erase sample loading history. (2) Immediately after the pre-shear step, the sample was allowed to equilibrate at 25° C. for 10 minutes. (3) The sample was then subjected to a 1-100 s⁻¹ shear rate ramp during which 20 logarithmically-spaced data points were recorded at an interval of 30 s per point. (4) The sample was then immediately exposed to a downward shear rate ramp which had the same characteristics as the upward ramp, but was applied in the opposite direction (100-1 s⁻¹). The resulting viscosity versus shear rate curves were fit to a 2^{nd} log polynomial model: apparent viscosity=a·(shear rate)^{[b+c in (shear rate)]}, where a, b, and c are curve fitting constants. a describes the apparent viscosity at a shear rate of 1 s⁻¹ and has units of centipoise (cps). The relationship n=1+[b+c In(shear rate)] describes the non-Newtonian behavior of the material, including shear thinning (pseudoplastic) or shear thickening (dilatent) behavior, in a similar fashion to the flow behavior index of the Herschel-Bulkley and power law (Ostwald) models. The parameter b is unitless and the parameter c has the units [ln(s)]. The shear stress hysteresis area (HA) bounded by the upward and downward shear rate ramps was also recorded during each measurement.

### Example 11

### Preparation of High Oleic and Commodity Soy Material Blends with Improved Characteristics

### Modulation of Isolate Viscosity by Mixing Commodity and High Oleic Defatted Flake Prior to First Extraction

Isolate Prepared from a Mixture of Commodity and High Oleic Soy Flakes

2007 Crop commodity and high oleic soybeans were converted to defatted soy flakes via standard hexane extraction and controlled desolventization. The defatted soy flakes were used to make the isolates. 10% slurries were made using the defatted flakes for each variety (commodity and high oleic). The slurries were blended to vary the amounts of high oleic and commodity material in the resultant slurry.

The two flake slurries were mixed at commodity: high oleic ratios of 80:20, 60:40 and 40:60. The blended slurries were processed to isolates as shown in the diagram below.

1000 g of the flour mixture was dispersed in 10 L. tap water in a colloid mill for 15 min. A defoamer (4 g, Ross Chemicals Foamblast) was added to the mixture that was then heated to 90° F. and circulated in the colloid mill for 15 min. The slurry (1600 ml) was centrifuged at 3000 rpm for 15 min. The supernatant was retained and the spent flakes were resuspended in 0.96 L of tap water at 90° F. The suspension was dispersed with a Silverson L4RT-A mixer at 5500 rpm for 60 sec and the pH was then adjusted to 9.00 with 1N NaOH. The solution was then centrifuged at 3000 rpm for 15 min. The first supernatant was combined with the second and the pH was adjusted to 4.5 with 2N HCl. The mixture was centrifuged for 15 min at 3000 rpm. The supernatant was discarded and the centrifuged curd was slurried with an equal weight of tap water in the centrifuge bottle by manual shaking for two minutes. The mixture was centrifuged for 15 min at 3000 rpm and the supernatant was discarded.

The centrifuged solids were slurried with a sufficient amount of water to obtain a slurry containing approximately 14% solids. The solids content was determined with a CEM Labwave 9000 moisture analyzer. The mixture was then adjusted to pH 7.0 with 2N NaOH with stirring and the resulting inhomogeneous mixture was dispersed by mixing with the Silverson for 60 sec at 5500 rpm. The pH was checked and adjusted to 7.0. The solids were measured and adjusted to 10% with tap water.

Soybeans with an oleic acid content and a PUFA content between that of commodity and high oleic soybeans, described as mid oleic beans, were purchased from Asoya. The beans were converted to defatted soy flake via standard hexane extraction and controlled desolventization. The defatted flake was processed using the same method described above to obtain 10% mid oleic isolate in-process slurry and the freeze-dried isolate.

In-process rheological measurements were done at 10% solids after neutralization. Rheology measurements were made on the sample within 30 min, and the remaining sample was stirred and heated to 85′C in a beaker on hot plate. This temperature was maintained for 7 minutes after which the sample was immediately cooled by immersing it in an ice bath. The sample was then frozen and freeze dried.

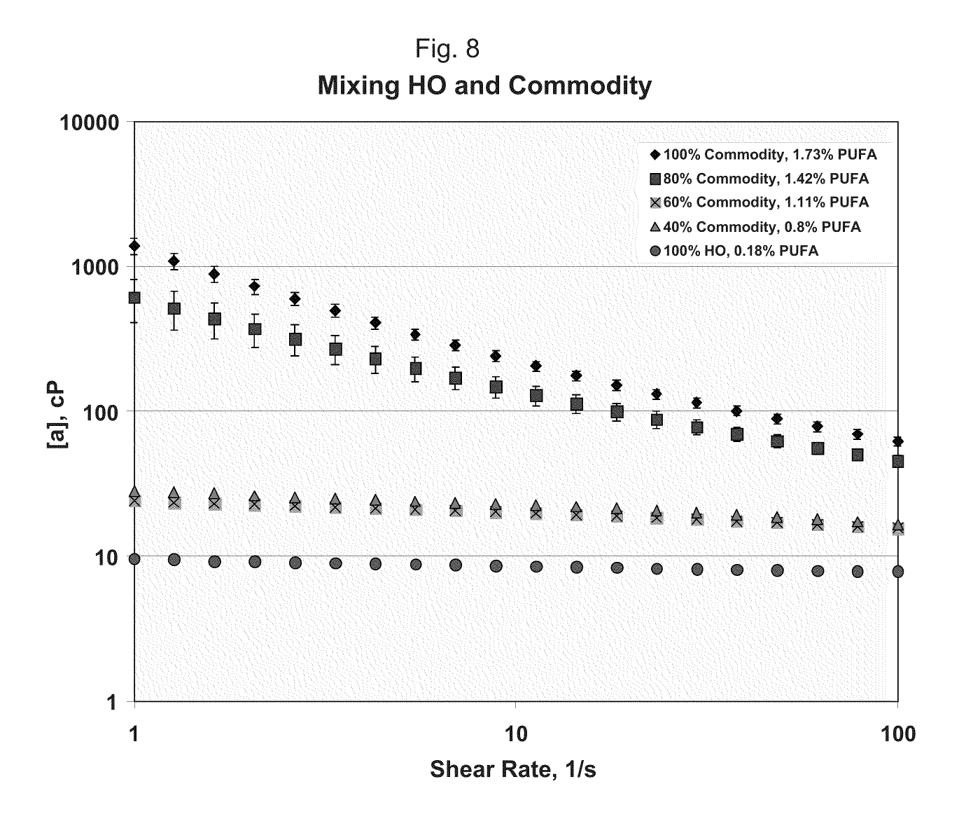

The results are shown in Table 4 and FIG. 8.

TABLE 4
Effect of mixing commodity and high oleic flake on the in process
viscosity, (‘a’ in cP at shear rate of 1/s)
of isolate slurry at 10% solids content.

| | | high | | |
|---|---|---|---|---|
| Sample | Commodity | oleic | % PUFA on flake | |
| Description | flake % | flake % | basis | a, cP |
| Commodity Control | 100% | 0% | 1.73 | 1387 |
| Com:HO::80:20 | 80% | 20% | 1.42 | 614.5 |
| Corn:HO::60:40 | 60% | 40% | 1.11 | 24.2 |
| Com:HO::40:60 | 40% | 60% | 0.80 | 28.2 |
| HO control | 0% | 100% | 0.18 | 11.2 |
| Mid Oleic Control | 0% | 0% | 0.73 | 106 |

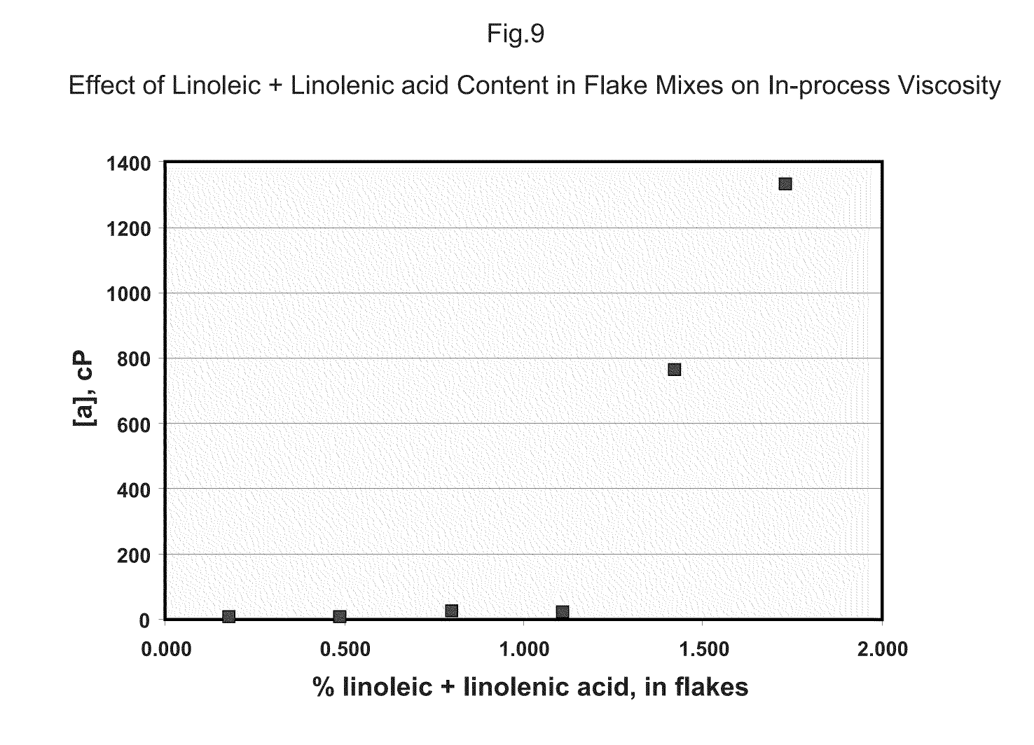

Even with inclusion of only 40% high oleic slurry, the viscosity dropped significantly, approximating the viscosity obtained with 100% high oleic slurries. This makes it possible to dry these blends at higher solids to dryer. Thus it is possible to obtain the functional and processing advantages of the more expensive 100% high oleic isolates when including certain levels of the less expensive commodity isolate. The ‘a’ values, i.e. viscosity at a shear rates of 1 reciprocal second were plotted as a function of linoleic and linolenic fatty acid content in the flake (calculated for the blends of the original fatty acid contents in the high oleic and commodity soy flake. The results are shown in FIG. 9. From the results it can be concluded that viscosity can be modulated by changing the fatty acid profile by blending flake with different fatty acid profiles. The above results show that isolates with varying viscosities can be produced by blending flakes with different polyunsaturated fatty acid profiles without using technologies, such as hydrolysis which add to the manufacturing cost and which produce a bitter flavor of the isolate. The isolates obtained from the blended flakes may be used for various applications ranging from products, such as, but not limited to soy milk, puddings, and gels to meat analogs.

### Example 12

### Modification of High Oleic Soy Protein Viscosity by Addition of Linoleic and Linolenic Acid Mixture to Defatted Flake Prior to First Extraction

Soy Protein Preparation from Commodity Soy Flake

2007 Crop commodity soybeans were converted to defatted soy flakes via standard hexane extraction and controlled desolventization. The flake (1000 g) was dispersed in 10 L tap water in a colloid mill for 15 min. A defoamer (4 g, Ross Chemicals Foamblast) was added to the mixture that was then heated to 90° F. and circulated in the colloid mill for 15 min. Two liter batches of the extract were centrifuged at 3000 rpm for 15 minutes.

The supernatant was retained and the spent flakes were re-suspended in 1.2 L of tap water at 90° F. The suspension was dispersed with a Silverson L4RT-A mixer at 5500 rpm for 60 sec and the pH was then adjusted to 9.00 with 1N NaOH. The solution was then centrifuged at 3000 rpm for 15 min. The first supernatant was combined with the second and the pH was adjusted to 4.5 with 2N HCl. The mixture was centrifuged for 15 min at 3000 rpm. The supernatant was discarded and the centrifuged curd was slurried with an equal weight of tap water in the centrifuge bottle by manual shaking for two minutes. The mixture was centrifuged for 15 min at 3000 rpm and the supernatant was discarded.

The centrifuged solids were slurried with a sufficient amount of water to obtain a slurry containing approximately 14% solids. The solids content was determined with a CEM Labwave 9000 moisture analyzer. The mixture was then adjusted to pH 7.0 with 2N NaOH with stirring and the resulting inhomogeneous mixture was dispersed by mixing with the Silverson for 60 sec at 5500 rpm. The pH was checked and adjusted to 7.0. The solids were measured and adjusted to 10% with tap water. Rheology measurements were made on the isolated soy protein within 30 min, and the remaining sample was stirred and heated to 85° C. in a beaker on hot plate. This temperature was maintained for 7 minutes after which the sample was immediately cooled by immersing it in an ice bath. The sample was then frozen and freeze dried.

Soy Protein Preparation from High Oleic Soy Flake

2007 Crop high oleic soy beans were converted to defatted soy flakes via standard hexane extraction and controlled desolventization. The flakes (1000 g) were dispersed in 10 L tap water in a colloid mill for 15 min. A defoamer (4 g, Ross Chemicals Foamblast) was added to the mixture that was then heated to 90° F. and circulated in the colloid mill for 15 min.

The slurry (1600 ml) was centrifuged at 3000 rpm for 15 min. The supernatant was retained and the spent flakes were resuspended in 0.96 L of tap water at 90° F. The suspension was dispersed with a Silverson L4RT-A mixer at 5500 rpm for 60 sec and the pH was then adjusted to 9.00 with 1N NaOH. The solution was then centrifuged at 3000 rpm for 15 min. The first supernatant was combined with the second and the pH was adjusted to 4.5 with 2N HCl. The mixture was centrifuged for 15 min at 3000 rpm. The supernatant was discarded and the centrifuged curd was slurried with an equal weight of tap water in the centrifuge bottle by manual shaking for two minutes. The mixture was centrifuged for 15 min at 3000 rpm and the supernatant was discarded.

The centrifuged solids were slurried with a sufficient amount of water to obtain a slurry containing approximately 14% solids. The solids content was determined with a CEM Labwave 9000 moisture analyzer. The mixture was then adjusted to pH 7.0 with 2N NaOH with stirring and the resulting inhomogeneous mixture was dispersed by mixing with the Silverson for 60 sec at 5500 rpm. The pH was checked and adjusted to 7.0. The solids were measured and adjusted to 10% with tap water.

Rheology measurements were made on the isolated soy protein within 30 min, and the remaining sample was stirred and heated to 85° C. in a beaker on hot plate. This temperature was maintained for 7 minutes after which the sample was immediately cooled by immersing it in an ice bath. The sample was then frozen and freeze dried.

Soy Protein Prepared from High Oleic Soy Rake Treated with Polyunsaturated Fatty Acids.

The flake (1000 g) was dispersed in 10 L tap water in a colloid mill for 15 min. A defoamer (4 g, Ross Chemicals Foamblast) was added to the mixture that was then heated to 90° F. and circulated in the colloid mill for 15 min. Four 2 L batches of slurry were prepared.

Linoleic acid (3.17 ml) and linolenic acid (0.41 ml) were added drop wise to a sample of slurry (200 ml) that was taken from one of the 2 L batches. The slurry/fatty acid mixture was blended with a Silverson mixer for 60 seconds to disperse the fatty acids. The 200 ml mixture was then combined with the other 1.8 L and the resulting mixture was homogenized in the colloid mill for 5 min. Two aliquots of 800 ml each were taken from the homogenized mixture and placed in 1 L vessels that were then placed in a Hanson Dissolution Station SR8Plus. The water bath of the Hanson was set at 50′C and the overhead stirrers were set at 120 rpm.

After the slurry was stirred for 30 min at 50° C., it was centrifuged at 3000 rpm for 15 min. The supernatant was retained and the spent flakes were resuspended in 0.96 L of tap water at 90° F. The suspension was dispersed with a Silverson L4RT-A mixer at 5500 rpm for 60 sec and the pH was then adjusted to 9.00 with 1N NaOH. The solution was then centrifuged at 3000 rpm for 15 min. The first supernatant was combined with the second and the pH was adjusted to 4.5 with 2N HCl. The mixture was centrifuged for 15 min at 3000 rpm. The supernatant was discarded and the centrifuged curd was slurried with an equal weight of tap water in the centrifuge bottle by manual shaking for two minutes. The mixture was centrifuged for 15 min at 3000 rpm and the supernatant was discarded.

The centrifuged solids were slurried with a sufficient amount of water to obtain a slurry containing approximately 14% solids. The solids content was determined with a CEM Labwave 9000 moisture analyzer. The mixture was then adjusted to pH 7.0 with 2N NaOH with stirring and the resulting inhomogeneous mixture was dispersed by mixing with the Silverson for 60 sec at 5500 rpm. The pH was checked and adjusted to 7.0. The solids were measured and adjusted to 10% with tap water.

Rheology measurements were made on the 10% in-process protein sample within 30 min, and the remaining sample was stirred and heated to 85° C. in a beaker on hot plate. This temperature was maintained for 7 minutes after which the sample was immediately cooled by immersing it in an ice bath. The sample was then frozen and freeze dried. Color measurements were made on the freeze-dried protein isolate.

This experiment was repeated with the following combinations of linoleic linolenic acid:

| Linoleic acid | + | Linolenic acid |
|---|---|---|
| 8.56 ml | | 1.11 ml |
| 4.28 ml | | 0.554 ml |
| 0.797 ml | | 0.103 ml |
| 0.318 ml | | 0.041 ml |
| 0.159 ml | | 0.021 ml |
| 0.066 ml | | 0.008 ml |
| 0 ml | | 0 ml |

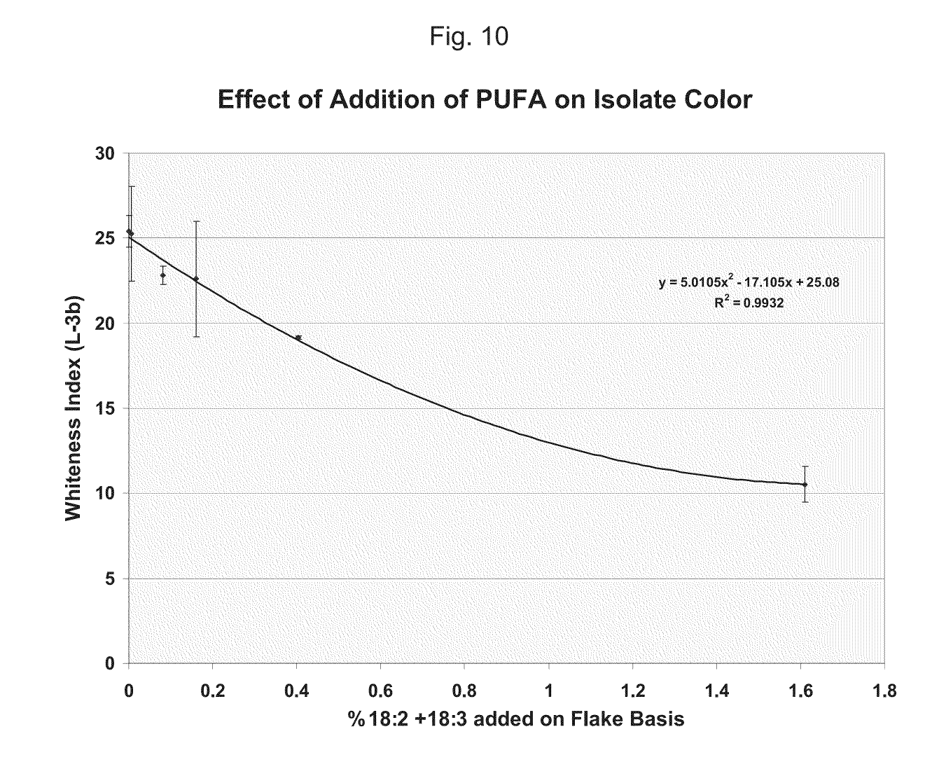

The effect of the added PUFAs on the in-process viscosity and the whiteness index of the dried isolate are shown in the attached Table 5. and FIG. 10.

TABLE 5

| | | | | Total | | | Total | | |
|---|---|---|---|---|---|---|---|---|---|
| | | | | Added | Total | Total | Linoleic + | | |
| | | Added | Added | Linoleic + | Linoleic | Linolenic | Linolenic | | |
| | | Linoleic | Linolenic | Linolenic | acid in | acid in | Acid in | | |
| | Incuba- | acid | acid | Acid | extraction | extraction | Extraction | FAH | |
| Flake | tion | (g/100 g | (g/100 g | (g/100 g | (g/100 g | (g/100 g | (g/100 g | % | a |
| (2007) | Time | flake) | flake) | flake) | flakes) | flake) | flake) | (flake) | (cP) |
| Cm¹ | None | 0.000 | 0.000 | 0.000 | 1.465 | 0.187 | 1.652 | 3.26 | 1903 |
| HO² | None | 0.000 | 0.000 | 0.000 | 0.120 | 0.047 | 0.167 | 2.59 | 12 |
| HO² | 30 min | 0.000 | 0.000 | 0.000 | 0.120 | 0.047 | 0.167 | 2.59 | 19 |
| HO² | 30 min | 3.350 | 0.500 | 3.850 | 3.470 | 0.547 | 4.017 | 2.59 | 3702 |
| HO² | 30 min | 1.925 | 0.250 | 2.175 | 2.045 | 0.297 | 2.342 | 2.59 | 2631 |
| HO² | 30 min | 1.430 | 0.185 | 1.615 | 1.550 | 0.232 | 1.782 | 2.59 | 3311 |
| HO² | 30 min | 1.073 | 0.139 | 1.211 | 1.193 | 0.186 | 1.378 | 2.59 | 2336 |
| HO² | 30 min | 0.358 | 0.046 | 0.404 | 0.478 | 0.093 | 0.571 | 2.59 | 1091 |
| HO² | 30 min | 0.143 | 0.018 | 0.161 | 0.263 | 0.065 | 0.328 | 2.59 | 521 |
| HO² | 30 min | 0.072 | 0.009 | 0.081 | 0.192 | 0.056 | 0.248 | 2.59 | 30 |
| HO² | 30 min | 0.029 | 0.037 | 0.066 | 0.149 | 0.084 | 0.233 | 2.59 | 20 |

¹Cm = commodity
²HO = high oleic

### Example 13

### Effect of Individual Fatty Acids on High Oleic Soy Protein Isolate Viscosity

The experiment was repeated by adding 3.23 g of each of the following individual fatty acids to 2 L of the first extract as described in Example 12. The stearic acid was dissolved in 20 ml ethanol that was added dropwise to 200 ml of the slurry being blended with the Silverson. The other acids were added directly as pure liquids.
Stearic acid (3.23 g)
Oleic acid (3.23 g)
Linoleic acid (3.23 g)
Linolenic acid (3.23 g)
Linoleic acid (2.86 g) Linolenic acid (0.36 g)

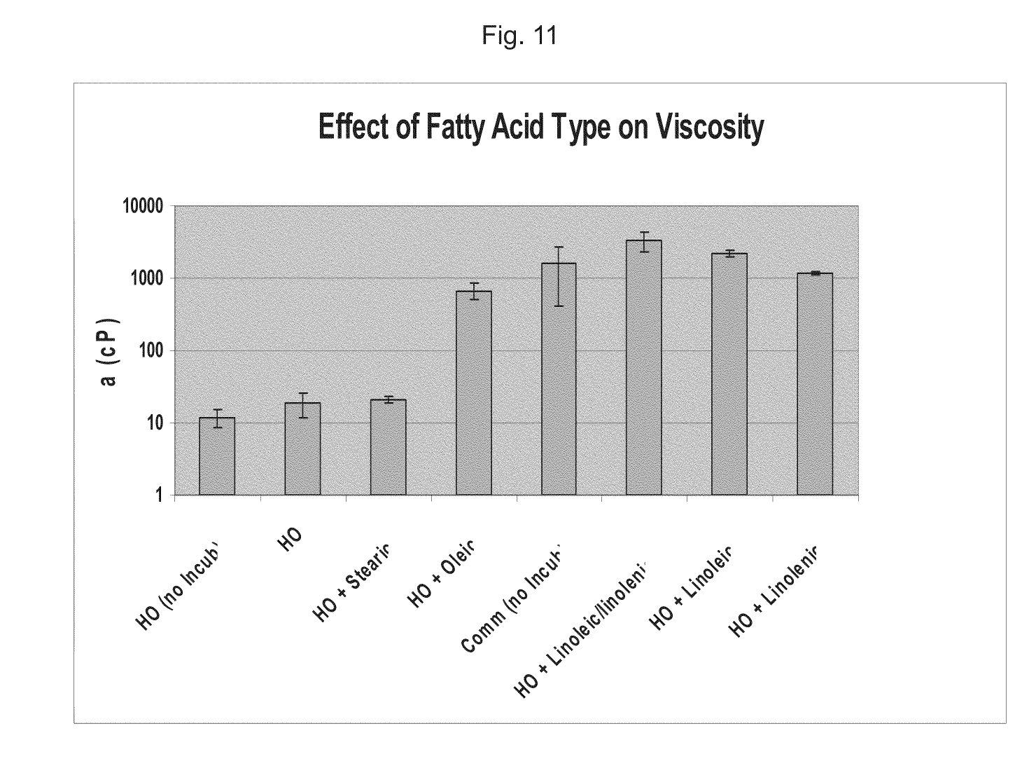

The results are shown in Table 6 and FIG. 11. The saturated stearic acid has no effect on the viscosity of the isolated protein, the monounsaturated oleic has some effect and the polyunsaturated linoleic and linolenic acids have a greater effect. The mixture of linoleic and linolenic acids has a greater effect than either linoleic acid or linolenic acid has on its own.

TABLE 6

| Sample | Mean a (cP) | stdev (cP) |
|---|---|---|
| 2007 HO (w/o incubation) | 11.9 | 3.3 |
| 2007 HO | 18.8 | 7.0 |
| 2007 HO + Stearic | 20.8 | 2.0 |
| 2007 HO + Oleic | 670 | 174 |
| 2007 Comm (no Incub) | 1576 | 1174 |
| 2007 HO + Linoleic/linolenic | 3310 | 1012 |
| 2007 HO + Linoleic | 2199 | 186 |
| 2007 HO + Linolenic | 1153 | 69 |

### Example 14

### Hunter Color Determination of Soybean Protein Products

Color measurements using the Hunter colorimeter can be made on soy protein powders, such as high oleic protein and commodity protein powders and 5% aqueous slurries. Two units of L value differences can be detected visually and one unit of Whiteness index differences can be detected. The whiteness indeces between the various protein products of the invention can be compared. Whiteness index measurements of for example 5% by weight solids samples of the suspension for the isolates can be determined using a HunterLab Labscan XE colorimeter manufactured by Hunter Associates Laboratory (HunterLab, Reston, Va.). For the whiteness index measurement, protein samples are dispersed on a 5% w/w basis: (5.25 g) is added to deionized water (100 mL). The results obtained using the Hunter Colorimeter are reported in units of L, a, and b. Whiteness Index is calculated from the L and b scale values using the following: Whiteness Index=L−3b.

### Example 15

### Gel Strength Measurements of Soy Protein Isolates

Gels can be prepared by the addition of NaCl and mixing for several minutes with additional scraping every 30 seconds. Following preparation, gels are packed into 5 mL glass vials using a disposable cartridge mini gun dispenser. Care has to be taken to eliminate any residual air bubbles. Vials are sealed with tightly crimped septum and cap. Sealed vials are either placed immediately in the refrigerator and stored for 16-24 hours (refrigerated gel) or incubated in an 80° C. bath for 30 minutes, cooled for 30 minutes in a 25° C. water bath prior to refrigeration for 16-24 hrs (pasteurized gel).

Gel strength is measured either on a texture Analyzer (TAXT.2i, Stable Micro Systems, UK) or an an AR-1000 Rheometer (TA Instruments). When gel strength is measured on the texture Analyzer, gels are removed from the refrigerator and warmed in a 25° C. Decapped sample vials are centered on the loading platform and a 3 mm diameter stainless cylinder punch probe is used for measurement. Gels are penetrated twice in the center of the vial to a depth of 10 mm and the data recorded using the instrument manufacturer's software. The area under the positive portion of the curve is integrated and recorded (labeled area). Gel preparation and measurements are replicated on a second day and the data averaged and recorded.

### Example 16

### Improvement of Drying Efficiency by Using High Oleic Soybeans

The blended protein products of the invention can be fed to a pasteurizer or a dryer at higher feed solids compared to for example commodity soy protein products. This can be explained by the reduced viscosity of some of the blended soy protein products of the invention (with 0.17% to about 1.65% PUFAS combined in the final protein product). When protein products are fed to a pasteurizer or a dryer at increased feed solids, less water has to be removed in every pound fed to the dryer resulting in decreased energy costs, and more solids can be dried per hour resulting in better capital utilization as well as higher production quantities.

### Example 17

### Fatty Acid Analysis of Soybean Isolates

The relative amounts of the fatty acids of isolated soy protein can be determined as follows. The isolated soy protein can be extracted by the acid hydrolysis fat method (AOAC 922.06). Extracted lipid was saponified with alcoholic sodium hydroxide. The fatty acids was esterified in methanol, with boron trifluoride as a catalyst, taken up in heptane, and injected on an Agilent 5890 Gas Chromatograph equipped with a flame ionization detector and cool on-column injector. Fatty acid methyl esters were separated on a Supelco SP-2560 column (100 m×0.25 mm ID). The column oven temperature was set to 140° C. for 5 minutes, then heated at 4° C. per minute to a maximum temperature of 240° C. and held at that temperature to the end of the analysis. The percent of individual fatty acid methyl esters were calculated from a set of standards containing known concentrations of prepared methyl esters of selected fatty acids.

What is claimed is:

1-21. (canceled)

22. A first soy protein product obtained from commodity soybean defatted flakes and at least one other different soybean defatted flake with a combined PUFA content of about 0.17% to about 1.65% by weight, wherein said first product, has at least one characteristic selected from the group consisting of: reduced viscosity, improved drying efficiency and improved whiteness index when compared to a second soy protein product obtained from the commodity soybean defatted flakes, and further wherein the same process is used to produce the first and second protein product.

23. The first soy protein product of claim 22, wherein the viscosity of the first soy protein product is reduced by at least 25%.

24. The first soy protein product of claim 22 wherein the drying efficiency of the first soy protein product is increased by at least 1%.

25. The first soy protein product of claim 22 wherein the whiteness index of the first soy protein product is increased by at least 2.5%.

26. The first soy protein product of claim 22 wherein said protein product has at least 40% protein (N×6.25) on a moisture-free basis.

27. The first soy protein product of claim 22 wherein said protein product has at least 65% protein (N×6.25) on a moisture-free basis. 28. The first soy protein product of claim 22 wherein said protein product has at least 90% protein (N×6.25) on a moisture-free basis.

28. The first soy protein product of claim 22 wherein said product is selected from the group consisting of a soy protein isolate, a soy protein concentrate, soy meal, full fat flour, soymilk powder, defatted flour, soymilk, textured proteins, textured flours, textured concentrates and textured isolates.

29. A food which has incorporated therein the first soy protein product of claim 22.

30. A beverage which has incorporated therein the first soy protein product of claim 22.

31. Animal feed which has incorporated therein the first soy protein product of claim 22.

32. A method for improving drying efficiency of the soy protein product of claim 22, comprising:
a) feeding a first soy protein product obtained from commodity soybean defatted flakes and at least one other different soybean defatted flake at higher feed solids to a pasteurizer or a dryer, compared to feeding a second soy protein product obtained from the commodity soybean defatted flakes; and
b) measuring improved drying efficiency of the soy protein product.

33. A method of preparing a soy protein product of claim 22 with an increased whiteness index, comprising:
a) obtaining defatted flakes from a first commodity soybean and from a second soybean with a combined PUFA content of 0.17% to about 1.65% by weight; and
b) measuring increased whiteness.

34. A method of preparing the soy protein product of claim 22 with a reduced viscosity, comprising:
a) obtaining defatted flakes from a first commodity soybean and from a second soybean with a combined PUFA content of about 0.17% to about 1.65% by weight; and
b) measuring reduced viscosity.

35. A first soy protein product obtained from commodity soybean defatted flakes and at least one other different soybean defatted flake with a combined PUFA content of more than 1.65% by weight, wherein said first product, has at least one characteristic selected from group consisting of: increased viscosity and increased gel strength, when compared to a second soy protein product obtained from the other different soybean defatted flakes, and further wherein the same process is used to produce the first and second protein product.

36. A first soy protein product obtained from high oleic soybean defatted flakes, obtained by the method comprising of:
a) preparing defatted flakes from a high oleic soybean;
b) adding PUFAs; and
c) measuring the viscosity of the soy protein product;
wherein said first soy protein product has increased viscosity when compared to a second soy protein product obtained from high oleic soybean defatted flakes without addition of PUFAs, and further wherein the same process is used to produce the first and second protein product.

37. The first soy protein product of claim 36, wherein the added PUFA content is about 0.066% to about 3.85% by weight.


SEQUENCE LISTING (26 sequences)

SEQ ID NO: 1 - DNA, 7993 bp, Artificial Sequence
  recombinant DNA fragment PHP21676A
cgcgccaagcttggatccgcgccaagcttggatcctagaactagaaacgtgatgccacttgttattgaagtcgattacagcatctattctgttttactatttataactttgccatttctgacttttgaaaactatctctggatttcggtatcgctttgtgaagatcgagcaaaagagacgttttgtggacgcaatggtccaaatccgttctacatgaacaaattggtcacaatttccactaaaagtaaataaatggcaagttaaaaaaggaatatgcattttactgattgcctaggtgagctccaagagaagttgaatctacacgtctaccaaccgctaaaaaaagaaaaacattgatatgtaacctgattccattagcttttgacttcttcaacagattctctacttagatttctaacagaaatattattactagcacatcattttcagtctcactacagcaaaaaatccaacggcacaatacagacaacaggagatatcagactacagagatagatagatgctactgcatgtagtaagttaaataaaaggaaaataaaatgtcttgctaccaaaactactacagactatgatgctcaccacaggccaaatcctgcaactaggacagcattatcttatatatattgtacaaaacaagcatcaaggaacatttggtctaggcaatcagtacctcgttctaccatcaccctcagttatcacatccttgaaggatccattactgggaatcatcggcaacacatgctcctgatggggcacaatgacatcaagaaggtaggggccaggggtgtccaacattctctgaattgccgctctaagctcttccttcttcgtcactcgcgctgccggtatcccacaagcatcagcaaacttgagcatgtttgggaatatctcgctctcgctagacggatctccaagataggtgtgagctctattggacttgtagaacctatcctccaactgaaccaccatacccaaatgctgattgttcaacaacaatatcttaactgggagattctccactcttatagtggccaactcctgaacattcatgatgaaactaccatccccatcaatgtcaaccacaacagccccagggttagcaacagcagcaccaatagccgcaggcaatccaaaacccatggctccaagaccccctgaggtcaaccactgcctcggtctcttgtacttgtaaaactgcgcagcccacatttgatgctgcccaaccccagtactaacaatagcatctccattagtcaactcatcaagaacctcgatagcatgctgcggagaaatcgcgtcctggaatgtcttgtaacccaatggaaacttgtgtttctgcacattaatctcttctctccaacctccaagatcaaacttaccctccactcctttctcctccaaaatcatattaattcccttcaaggccaacttcaaatccgcgcaaaccgacacgtgcgcctgcttgttcttcccaatctcggcagaatcaatatcaatgtgaacaatcttagccctactagcaaaagcctcaagcttcccagtaacacggtcatcaaaccttaccccaaaggcaagcaacaaatcactattgtcaacagcatagttagcataaacagtaccatgcatacccagcatctgaagggaatattcatcaccaataggaaaagttccaagacccattaaagtgctagcaacgggaataccagtgagttcaacaaagcgcctcaattcagcactggaattcaaactgccaccgccgacgtagagaacgggcttttgggcctccatgatgagtctgacaatgtgttccaattgggcctcggcggggggcctgggcagcctggcgaggtaaccggggaggttaacgggctcgtcccaattaggcacggcgagttgctgctgaacgtctttgggaatgtcgatgaggaccggaccggggcggccggaggtggcgacgaagaaagcctcggcgacgacgcgggggatgtcgtcgacgtcgaggatgaggtagttgtgcttcgtgatggatctgctcacctccacgatcggggtttcttggaaggcgtcggtgccgatcatccggcgggcgacctggccggtgatggcgacgactgggacgctgtccattaaagcgtcggcgaggccgctcacgaggttggtggcgccggggccggaggtggcaatgcagacgccggggaggccggaggaacgcgcgtagccttcggcggcgaagacgccgccctgctcgtggcgcgggagcacgttgcggatggcggcggagcgcgtgagcgcctggtggatctccatcgacgcaccgccggggtacgcgaacaccgtcgtcacgccctgcctctccagcgcctccacaaggatgtccgcgcccttgcgaggttcgccggaggcgaaccgtgacacgaagggctccgtggtcggcgcttccttggtgaagggcgccgccgtggggggtttggagatggaacatttgattttgagagcgtggttgggtttggtgagggtttgatgagagagagggagggtggatctagtaatgcgtttggggaaggtggggtgtgaagaggaagaagagaatcgggtggttctggaagcggtggccgccattgtgttgtgtggcatggttatacttcaaaaactgcacaacaagcctagagttagtacctaaacagtaaatttacaacagagagcaaagacacatgcaaaaatttcagccataaaaaaagttataatagaatttaaagcaaaagtttcattttttaaacatatatacaaacaaactggatttgaaggaagggattaattcccctgctcaaagtttgaattcctattgtgacctatactcgaataaaattgaagcctaaggaatgtatgagaaacaagaaaacaaaacaaaactacagacaaacaagtacaattacaaaattcgctaaaattctgtaatcaccaaaccccatctcagtcagcacaaggcccaaggtttattttgaaataaaaaaaaagtgattttatttctcataagctaaaagaaagaaaggcaattatgaaatgatttcgactagatctgaaagtccaacgcgtattccgcagatattaaagaaagagtagagtttcacatggatcctagatggacccagttgaggaaaaagcaaggcaaagcaaaccagaagtgcaagatccgaaattgaaccacggaatctaggatttggtagagggagaagaaaagtaccttgagaggtagaagagaagagaagagcagagagatatatgaacgagtgtgtcttggtctcaactctgaagcgatacgagtttagaggggagcattgagttccaatttatagggaaaccgggtggcaggggtgagttaatgacggaaaagcccctaagtaacgagattggattgtgggttagattcaaccgtttgcatccgcggcttagattggggaagtcagagtgaatctcaaccgttgactgagttgaaaattgaatgtagcaaccaattgagccaaccccagcctttgccctttgattttgatttgtttgttgcatactttttatttgtcttctggttctgactctctttctctcgtttcaatgccaggttgcctactcccacaccactcacaagaagattctactgttagtattaaatattttttaatgtattaaatgatgaatgcttttgtaaacagaacaagactatgtctaataagtgtcttgcaacattttttaagaaattaaaaaaaatatatttattatcaaaatcaaatgtatgaaaaatcatgaataatataattttatacatttttttaaaaaatcttttaatttcttaattaatatcttaaaaataatgattaatatttaacccaaaataattagtatgattggtaaggaagatatccatgttatgtttggatgtgagtttgatctagagcaaagcttactagagtcgaccgatccgtcgacggcgcggatcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccggagctggtcatctcgctcatcgtcgagtcggcggccggagctggtcatctcgctcatcgtcgagtcggcggccgccggtcctctctctttccgtggcatggcaatctattgggctgtccagggttgcatccttactggtgtttgggtcattgcccatgagtgtggtcaccatgcattcagtgactaccagctgcttgatgatattgttggccttatcctccactccgctctcctagtcccgtacttttcatggaaatacagccatcgccgtcaccactccaacactggttctcttgagcgggatgaagtatttgtgccaaagcagaagtcctgtatcaagtggtactctaaataccttaacaatcctccaggcagagtcctcactcttgctgtcaccctcacacttggttggcccttgtacttggctttaaatgtttctggaaggccttatgatagatttgcttgccactatgacccatatggtcccatttactctgatcgtgaacgacttcaaatatatatatcagatgcaggagtacttgcaggacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattatgggattctgaacaaggtgtttcatcacataactgatactcatgtggctcaccatctcttctctacaatgccacattaccatgcaatggaggcaaccaatgcaatcaagccaatattgggtgagtactaccaatttgatgacacaccattttacaaggcactgtggagagaagcgagagagtgcctctatgtggagccagatgaaggaacatccgagaagggctcctccaccgtttaagattgcagaaatcagagcttcaataccaaaacattgctgggtcaagaatccatggagatccctcagttatgttctcagggatgtgcttgtaattgctgcattggtggctgcagcaattcacttcgacaactggcttctctggctaatctattgccccattcaaggcacaatgttctgggctctctttgttcttggacatgattgtggccatggaagcttttcagatagccctttgctgaatagcctggtgggacacatcttgcattcctcaattcttgtgccataccatggatggagaattagccacagaactcaccatcaaaaccatggacacattgagaaggatgagtcatgggttccattaacagagaagatttacaagaatctagacagcatgacaagactcattagattcactgtgccatttccattgtttgtgtatccaatttatttgttttcaagaagccccggaaaggaaggctctcacttcaatccctacagcaatctgttcccacccagtgagagaaaaggaatagcaatatcaacactgtgttgggctaccatgttttctctgcttatctatctctcattcataactagtccacttctagtgctcaagctctatgggcggccgccgactcgacgatgagcgagatgaccagctccggccgccgactcgacgatgagcgagatgaccagctccggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccgtcgacgg

SEQ ID NO: 2 - DNA, 1533 bp, Artificial sequence
  recombinant DNA fragment
cggtcctctctctttccgtggcatggcaatctattgggctgtccagggttgcatccttactggtgtttgggtcattgcccatgagtgtggtcaccatgcattcagtgactaccagctgcttgatgatattgttggccttatcctccactccgctctcctagtcccgtacttttcatggaaatacagccatcgccgtcaccactccaacactggttctcttgagcgggatgaagtatttgtgccaaagcagaagtcctgtatcaagtggtactctaaataccttaacaatcctccaggcagagtcctcactcttgctgtcaccctcacacttggttggcccttgtacttggctttaaatgtttctggaaggccttatgatagatttgcttgccactatgacccatatggtcccatttactctgatcgtgaacgacttcaaatatatatatcagatgcaggagtacttgcaggacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattatgggattctgaacaaggtgtttcatcacataactgatactcatgtggctcaccatctcttctctacaatgccacattaccatgcaatggaggcaaccaatgcaatcaagccaatattgggtgagtactaccaatttgatgacacaccattttacaaggcactgtggagagaagcgagagagtgcctctatgtggagccagatgaaggaacatccgagaagggctcctccaccgtttaagattgcagaaatcagagcttcaataccaaaacattgctgggtcaagaatccatggagatccctcagttatgttctcagggatgtgcttgtaattgctgcattggtggctgcagcaattcacttcgacaactggcttctctggctaatctattgccccattcaaggcacaatgttctgggctctctttgttcttggacatgattgtggccatggaagcttttcagatagccctttgctgaatagcctggtgggacacatcttgcattcctcaattcttgtgccataccatggatggagaattagccacagaactcaccatcaaaaccatggacacattgagaaggatgagtcatgggttccattaacagagaagatttacaagaatctagacagcatgacaagactcattagattcactgtgccatttccattgtttgtgtatccaatttatttgttttcaagaagccccggaaaggaaggctctcacttcaatccctacagcaatctgttcccacccagtgagagaaaaggaatagcaatatcaacactgtgttgggctaccatgttttctctgcttatctatctctcattcataactagtccacttctagtgctcaagctctatgg

SEQ ID NO: 3 - DNA, 28 bp, Artificial sequence
  primer
gcggccgccggtcctctctctttccgtg

SEQ ID NO: 4 - DNA, 31 bp, Artificial sequence
  primer
taaacggtggaggagcccttctcggatgttc

SEQ ID NO: 5 - DNA, 33 bp, Artificial sequence
  primer
gaacatccgagaagggctcctccaccgtttaag

SEQ ID NO: 6 - DNA, 28 bp, Artificial sequence
  primer
gcggccgcccatagagcttgagcactag

SEQ ID NO: 7 - DNA, 890 bp, Artificial sequence
  recombinant DNA fragment KSFAD2-hybrid
cggtcctctctctttccgtggcatggcaatctattgggctgtccagggttgcatccttactggtgtttgggtcattgcccatgagtgtggtcaccatgcattcagtgactaccagctgcttgatgatattgttggccttatcctccactccgctctcctagtcccgtacttttcatggaaatacagccatcgccgtcaccactccaacactggttctcttgagcgggatgaagtatttgtgccaaagcagaagtcctgtatcaagtggtactctaaataccttaacaatcctccaggcagagtcctcactcttgctgtcaccctcacacttggttggcccttgtacttggctttaaatgtttctggaaggccttatgatagatttgcttgccactatgacccatatggtcccatttactctgatcgtgaacgacttcaaatatatatatcagatgcaggagtacttgcaggacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattatgggattctgaacaaggtgtttcatcacataactgatactcatgtggctcaccatctcttctctacaatgccacattaccatgcaatggaggcaaccaatgcaatcaagccaatattgggtgagtactaccaatttgatgacacaccattttacaaggcactgtggagagaagcgagagagtgcctctatgtggagccagatgaaggaacatccgagaaggg

SEQ ID NO: 8 - DNA, 28 bp, Artificial sequence
  primer
gcggccgccggtcctctctctttccgtg

SEQ ID NO: 9 - DNA, 30 bp, Artificial sequence
  primer
tagagagagtaagtcctgcaagtactcctg

SEQ ID NO: 10 - DNA, 30 bp, Artificial sequence
  primer
caggagtacttgcaggacttactctctcta

SEQ ID NO: 11 - DNA, 29 bp, Artificial sequence
  primer
gcggccggccccttctcggatgttccttc

SEQ ID NO: 12 - DNA, 2460 bp, Artificial sequence
  seed-specific gene expression-silencing cassette
ccaagcttggatcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccggagctggtcatctcgctcatcgtcgagtcggcggccggagctggtcatctcgctcatcgtcgagtcggcggccgccgactcgacgatgagcgagatgaccagctccggccgccgactcgacgatgagcgagatgaccagctccggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccgtcgacggcgcg

SEQ ID NO: 13 - DNA, 8966 bp, Artificial sequence
  plasmid pKS210
gatcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccggagctggtcatctcgctcatcgtcgagtcggcggccggagctggtcatctcgctcatcgtcgagtcggcggccgccgactcgacgatgagcgagatgaccagctccggccgccgactcgacgatgagcgagatgaccagctccggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccgtcgacggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccaagcttggatccgcgccaagcttggatcctagaactagaaacgtgatgccacttgttattgaagtcgattacagcatctattctgttttactatttataactttgccatttctgacttttgaaaactatctctggatttcggtatcgctttgtgaagatcgagcaaaagagacgttttgtggacgcaatggtccaaatccgttctacatgaacaaattggtcacaatttccactaaaagtaaataaatggcaagttaaaaaaggaatatgcattttactgattgcctaggtgagctccaagagaagttgaatctacacgtctaccaaccgctaaaaaaagaaaaacattgatatgtaacctgattccattagcttttgacttcttcaacagattctctacttagatttctaacagaaatattattactagcacatcattttcagtctcactacagcaaaaaatccaacggcacaatacagacaacaggagatatcagactacagagatagatagatgctactgcatgtagtaagttaaataaaaggaaaataaaatgtcttgctaccaaaactactacagactatgatgctcaccacaggccaaatcctgcaactaggacagcattatcttatatatattgtacaaaacaagcatcaaggaacatttggtctaggcaatcagtacctcgttctaccatcaccctcagttatcacatccttgaaggatccattactgggaatcatcggcaacacatgctcctgatggggcacaatgacatcaagaaggtaggggccaggggtgtccaacattctctgaattgccgctctaagctcttccttcttcgtcactcgcgctgccggtatcccacaagcatcagcaaacttgagcatgtttgggaatatctcgctctcgctagacggatctccaagataggtgtgagctctattggacttgtagaacctatcctccaactgaaccaccatacccaaatgctgattgttcaacaacaatatcttaactgggagattctccactcttatagtggccaactcctgaacattcatgatgaaactaccatccccatcaatgtcaaccacaacagccccagggttagcaacagcagcaccaatagccgcaggcaatccaaaacccatggctccaagaccccctgaggtcaaccactgcctcggtctcttgtacttgtaaaactgcgcagcccacatttgatgctgcccaaccccagtactaacaatagcatctccattagtcaactcatcaagaacctcgatagcatgctgcggagaaatcgcgtcctggaatgtcttgtaacccaatggaaacttgtgtttctgcacattaatctcttctctccaacctccaagatcaaacttaccctccactcctttctcctccaaaatcatattaattcccttcaaggccaacttcaaatccgcgcaaaccgacacgtgcgcctgcttgttcttcccaatctcggcagaatcaatatcaatgtgaacaatcttagccctactagcaaaagcctcaagcttcccagtaacacggtcatcaaaccttaccccaaaggcaagcaacaaatcactattgtcaacagcatagttagcataaacagtaccatgcatacccagcatctgaagggaatattcatcaccaataggaaaagttccaagacccattaaagtgctagcaacgggaataccagtgagttcaacaaagcgcctcaattcagcactggaattcaaactgccaccgccgacgtagagaacgggcttttgggcctccatgatgagtctgacaatgtgttccaattgggcctcggcggggggcctgggcagcctggcgaggtaaccggggaggttaacgggctcgtcccaattaggcacggcgagttgctgctgaacgtctttgggaatgtcgatgaggaccggaccggggcggccggaggtggcgacgaagaaagcctcggcgacgacgcgggggatgtcgtcgacgtcgaggatgaggtagttgtgcttcgtgatggatctgctcacctccacgatcggggtttcttggaaggcgtcggtgccgatcatccggcgggcgacctggccggtgatggcgacgactgggacgctgtccattaaagcgtcggcgaggccgctcacgaggttggtggcgccggggccggaggtggcaatgcagacgccggggaggccggaggaacgcgcgtagccttcggcggcgaagacgccgccctgctcgtggcgcgggagcacgttgcggatggcggcggagcgcgtgagcgcctggtggatctccatcgacgcaccgccggggtacgcgaacaccgtcgtcacgccctgcctctccagcgcctccacaaggatgtccgcgcccttgcgaggttcgccggaggcgaaccgtgacacgaagggctccgtggtcggcgcttccttggtgaagggcgccgccgtggggggtttggagatggaacatttgattttgagagcgtggttgggtttggtgagggtttgatgagagagagggagggtggatctagtaatgcgtttggggaaggtggggtgtgaagaggaagaagagaatcgggtggttctggaagcggtggccgccattgtgttgtgtggcatggttatacttcaaaaactgcacaacaagcctagagttagtacctaaacagtaaatttacaacagagagcaaagacacatgcaaaaatttcagccataaaaaaagttataatagaatttaaagcaaaagtttcattttttaaacatatatacaaacaaactggatttgaaggaagggattaattcccctgctcaaagtttgaattcctattgtgacctatactcgaataaaattgaagcctaaggaatgtatgagaaacaagaaaacaaaacaaaactacagacaaacaagtacaattacaaaattcgctaaaattctgtaatcaccaaaccccatctcagtcagcacaaggcccaaggtttattttgaaataaaaaaaaagtgattttatttctcataagctaaaagaaagaaaggcaattatgaaatgatttcgactagatctgaaagtccaacgcgtattccgcagatattaaagaaagagtagagtttcacatggatcctagatggacccagttgaggaaaaagcaaggcaaagcaaaccagaagtgcaagatccgaaattgaaccacggaatctaggatttggtagagggagaagaaaagtaccttgagaggtagaagagaagagaagagcagagagatatatgaacgagtgtgtcttggtctcaactctgaagcgatacgagtttagaggggagcattgagttccaatttatagggaaaccgggtggcaggggtgagttaatgacggaaaagcccctaagtaacgagattggattgtgggttagattcaaccgtttgcatccgcggcttagattggggaagtcagagtgaatctcaaccgttgactgagttgaaaattgaatgtagcaaccaattgagccaaccccagcctttgccctttgattttgatttgtttgttgcatactttttatttgtcttctggttctgactctctttctctcgtttcaatgccaggttgcctactcccacaccactcacaagaagattctactgttagtattaaatattttttaatgtattaaatgatgaatgcttttgtaaacagaacaagactatgtctaataagtgtcttgcaacattttttaagaaattaaaaaaaatatatttattatcaaaatcaaatgtatgaaaaatcatgaataatataattttatacatttttttaaaaaatcttttaatttcttaattaatatcttaaaaataatgattaatatttaacccaaaataattagtatgattggtaaggaagatatccatgttatgtttggatgtgagtttgatctagagcaaagcttactagagtcgaccgatccgtcgacggcgcg

SEQ ID NO: 14 - DNA, 6611 bp, Artificial sequence
  plasmid PHP17731
cgcgcctatgcgggaccatcgcagcggacgagaagcggcacgagaacgcgtactcaagaatcgtggagaagcttctggaagtggaccccaccggggcaatggtggccatagggaacatgatggagaagaagatcacgatgccggcgcaccttatgtacgatggggatgaccccaggctattcgagcactactccgctgtggcgcagcgcataggcgtgtacaccgccaacgactacgcagacatcttggatttctcgttgacggtgaagattggagaagcttgaaggattgatgcctgaggggaagcgggccccaggatttccgtgtgtgggttgcccccgaggattaggaggttccaagaacgcgctgatgagcgagcgcgtaagatgaagaagcatcatgccgttaagttcagttggattttcaataaagaattgcttttgtgagcggccgccgactcgacgatgagcgagatgaccagctccggccgccgactcgacgatgagcgagatgaccagctccggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccgtcgacggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccaagcttggatcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccggagctggtcatctcgctcatcgtcgagtcggcggccggagctggtcatctcgctcatcgtcgagtcggcggccgctgagtgattgctcacgagtgtggtcaccatgccttcagcaagtaccaatgggttgatgatgttgtgggtttgacccttcactcaacacttttagtcccttatttctcatggaaaataagccatcgccgccatcactccaacacaggttcccttgaccgtgatgaagtgtttgtcccaaaaccaaaatccaaagttgcatggttttccaagtacttaaacaaccctctaggaagggctgtttctcttctcgtcacactcacaatagggtggcctatgtatttagccttcaatgtctctggtagaccctatgatagttttgcaagccactaccacccttatgctcccatatattctaaccgtgagaggcttctgatctatgtctctgatgttgctttgttttctgtgacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattaagcggccgcatgcctccagaaaagaaagaaattttcaagtccttggagggatgggcctcggagtgggtcctaccgctgctgaagcccgtggagcaatgctggcagccacaaaacttcctccctgacccctcccttccgcatgaagagttcagccatcaggtgaaggagcttcgcgaacgcactaaagagttacctgatgagtactttgtggtgctggtgggtgatatggtcaccgaggacgcgcttcccacttaccagaccatgatcaacaaccttgatggagtgaaagatgacagcggcacgagcccgagcccgtgggccgtgtggacccgggcctggaccgccgaggaaaacagacacggggatctgctcagaacttatttgtatctctctgggagggttgacatggctaaggtcgaaaagaccgtacattacctcatttcagctggcatggaccctgggacagacaacaacccatatttggggtttgtgtacacgtcattccaagagcgagcaacatttgtggcgcacgggaacacggctcggctcgcgaaggagggcggggatccagtgctggcgcg

SEQ ID NO: 15 - DNA, 4097 bp, Artificial sequence
  recombinant DNA fragment PHP17731A
cgcgccaagcttggatcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccggagctggtcatctcgctcatcgtcgagtcggcggccggagctggtcatctcgctcatcgtcgagtcggcggccgctgagtgattgctcacgagtgtggtcaccatgccttcagcaagtaccaatgggttgatgatgttgtgggtttgacccttcactcaacacttttagtcccttatttctcatggaaaataagccatcgccgccatcactccaacacaggttcccttgaccgtgatgaagtgtttgtcccaaaaccaaaatccaaagttgcatggttttccaagtacttaaacaaccctctaggaagggctgtttctcttctcgtcacactcacaatagggtggcctatgtatttagccttcaatgtctctggtagaccctatgatagttttgcaagccactaccacccttatgctcccatatattctaaccgtgagaggcttctgatctatgtctctgatgttgctttgttttctgtgacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattaagcggccgcatgcctccagaaaagaaagaaattttcaagtccttggagggatgggcctcggagtgggtcctaccgctgctgaagcccgtggagcaatgctggcagccacaaaacttcctccctgacccctcccttccgcatgaagagttcagccatcaggtgaaggagcttcgcgaacgcactaaagagttacctgatgagtactttgtggtgctggtgggtgatatggtcaccgaggacgcgcttcccacttaccagaccatgatcaacaaccttgatggagtgaaagatgacagcggcacgagcccgagcccgtgggccgtgtggacccgggcctggaccgccgaggaaaacagacacggggatctgctcagaacttatttgtatctctctgggagggttgacatggctaaggtcgaaaagaccgtacattacctcatttcagctggcatggaccctgggacagacaacaacccatatttggggtttgtgtacacgtcattccaagagcgagcaacatttgtggcgcacgggaacacggctcggctcgcgaaggagggcggggatccagtgctggcgcgcgcgcctatgcgggaccatcgcagcggacgagaagcggcacgagaacgcgtactcaagaatcgtggagaagcttctggaagtggaccccaccggggcaatggtggccatagggaacatgatggagaagaagatcacgatgccggcgcaccttatgtacgatggggatgaccccaggctattcgagcactactccgctgtggcgcagcgcataggcgtgtacaccgccaacgactacgcagacatcttggatttctcgttgacggtgaagattggagaagcttgaaggattgatgcctgaggggaagcgggccccaggatttccgtgtgtgggttgcccccgaggattaggaggttccaagaacgcgctgatgagcgagcgcgtaagatgaagaagcatcatgccgttaagttcagttggattttcaataaagaattgcttttgtgagcggccgccgactcgacgatgagcgagatgaccagctccggccgccgactcgacgatgagcgagatgaccagctccggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccgtcgacgg

SEQ ID NO: 16 - DNA, 3964 bp, Artificial sequence
  ALS selectable marker recombinant DNA fragment
ggtcgactctagtaagctttgctctagatcaaactcacatccaaacataacatggatatcttccttaccaatcatactaattattttgggttaaatattaatcattatttttaagatattaattaagaaattaaaagattttttaaaaaaatgtataaaattatattattcatgatttttcatacatttgattttgataataaatatattttttttaatttcttaaaaaatgttgcaagacacttattagacatagtcttgttctgtttacaaaagcattcatcatttaatacattaaaaaatatttaatactaacagtagaatcttcttgtgagtggtgtgggagtaggcaacctggcattgaaacgagagaaagagagtcagaaccagaagacaaataaaaagtatgcaacaaacaaatcaaaatcaaagggcaaaggctggggttggctcaattggttgctacattcaattttcaactcagtcaacggttgagattcactctgacttccccaatctaagccgcggatgcaaacggttgaatctaacccacaatccaatctcgttacttaggggcttttccgtcattaactcacccctgccacccggtttccctataaattggaactcaatgctcccctctaaactcgtatcgcttcagagttgagaccaagacacactcgttcatatatctctctgctcttctcttctcttctacctctcaaggtacttttcttctccctctaccaaatcctagattccgtggttcaatttcggatcttgcacttctggtttgctttgccttgctttttcctcaactgggtccatctaggatccatgtgaaactctactctttctttaatatctgcggaatacgcgttggactttcagatctagtcgaaatcatttcataattgcctttctttcttttagcttatgagaaataaaatcactttttttttatttcaaaataaaccttgggccttgtgctgactgagatggggtttggtgattacagaattttagcgaattttgtaattgtacttgtttgtctgtagttttgttttgttttcttgtttctcatacattccttaggcttcaattttattcgagtataggtcacaataggaattcaaactttgagcaggggaattaatcccttccttcaaatccagtttgtttgtatatatgtttaaaaaatgaaacttttgctttaaattctattataactttttttatggctgaaatttttgcatgtgtctttgctctctgttgtaaatttactgtttaggtactaactctaggcttgttgtgcagtttttgaagtataaccatgccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcgcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagttggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgattgcctagaccaaatgttccttgatgcttgttttgtacaatatatataagataatgctgtcctagttgcaggatttggcctgtggtgagcatcatagtctgtagtagttttggtagcaagacattttattttccttttatttaacttactacatgcagtagcatctatctatctctgtagtctgatatctcctgttgtctgtattgtgccgttggattttttgctgtagtgagactgaaaatgatgtgctagtaataatatttctgttagaaatctaagtagagaatctgttgaagaagtcaaaagctaatggaatcaggttacatatcaatgtttttctttttttagcggttggtagacgtgtagattcaacttctcttggagctcacctaggcaatcagtaaaatgcatattccttttttaacttgccatttatttacttttagtggaaattgtgaccaatttgttcatgtagaacggatttggaccattgcgtccacaaaacgtctcttttgctcgatcttcacaaagcgataccgaaatccagagatagttttcaaaagtcagaaatggcaaagttataaatagtaaaacagaatagatgctgtaatcgacttcaataacaagtggcatcacgtttctagttctagacccgggtac

SEQ ID NO: 17 - protein, 656 aa, Glycine max
MPHNTMAATASRTTRFSSSSSHPTFPKRITRSTLPLSHQTLTKPNHALKIKCSISKPPTAAPFTKEAPTTEPFVSRFASGEPRKGADILVEALERQGVTTVFAYPGGASMEIHQALTRSAAIRNVLPRHEQGGVFAAEGYARSSGLPGVCIATSGPGATNLVSGLADALMDSVPVVAITGQVARRMIGTDAFQETPIVEVSRSITKHNYLILDVDDIPRVVAEAFFVATSGRPGPVLIDIPKDVQQQLAVPNWDEPVNLPGYLARLPRPPAEAQLEHIVRLIMEAQKPVLYVGGGSLNSSAELRRFVELTGIPVASTLMGLGTFPIGDEYSLQMLGMHGTVYANYAVDNSDLLLAFGVRFDDRVTGKLEAFASRAKIVHIDIDSAEIGKNKQAHVSVCADLKLALKGINMILEEKGVEGKFDLGGWREEINVQKHKFPLGYKTFQDAISPQHAIEVLDELTNGDAIVSTGVGQHQMWAAQFYKYKRPRQWLTSGGLGAMGFGLPAAIGAAVANPGAVVVDIDGDGSFIMNVQELATIRVENLPVKILLLNNQHLGMVVQLEDRFYKSNRAHTYLGDPSSESEIFPNMLKFADACGIPAARVTKKEELRAAIQRMLDTPGPYLLDVIVPHQEHVLPMIPSNGSFKDVITEGDGRTRY

SEQ ID NO: 18 - protein, 4 aa, artificial sequence
  subsequence B
GQVP

SEQ ID NO: 19 - protein, 10 aa, artificial sequence
  subsequence F
GMVXQWEDRF

SEQ ID NO: 20 - protein, 5 aa, artificial sequence
  cloning site
MPHNT

SEQ ID NO: 21 - DNA, 6547 bp, artificial sequence
  plasmid PHP17064
gggcgaattgggttacccggaccggaattcgggatctgagtctagaaatccgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatgatcctatgcgtatggtatgacgtgtgttcaagatgatgacttcaaacctacctatgacgtatggtatgaacgtgtgtcgactgatgacttagatccactcgagcggctataaatacgtacctacgcaccctgcgctaccatccctagagctgcagcttatttttacaacaattaccaacaacaacaaacaacaaacaacattacaattactatttacaattacagtcgacccgtacccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcgcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagttggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgattgcctagaccaaatgttccttgatgcttgttttgtacaatatatataagataatgctgtcctagttgcaggatttggcctgtggtgagcatcatagtctgtagtagttttggtagcaagacattttattttccttttatttaacttactacatgcagtagcatctatctatctctgtagtctgatatctcctgttgtctgtattgtgccgttggattttttgctgtagtgagactgaaaatgatgtgctagtaataatatttctgttagaaatctaagtagagaatctgttgaagaagtcaaaagctaatggaatcaggttacatatcaatgtttttctttttttagcggttggtagacgtgtagattcaacttctcttggagctcacctaggcaatcagtaaaatgcatattccttttttaacttgccatttatttacttttagtggaaattgtgaccaatttgttcatgtagaacggatttggaccattgcgtccacaaaacgtctcttttgctcgatcttcacaaagcgataccgaaatccagagatagttttcaaaagtcagaaatggcaaagttataaatagtaaaacagaatagatgctgtaatcgacttcaataacaagtggcatcacgtttctagttctagacccgggtctagagtcgacctgcaggcatgcccgcggatatcgatgggccccggccgaagcttcggtccgggtcacccagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttcgataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgcctataaatgggctcgcgataatgtcggccaatcaggtccgacaatctatcgattgtatgggaagcccgatgcgccagacttgtttctgaaacatggcaaaggtagccttgccaatgatgttacagatgagatggtcagactaaactgcctgacggaatttatgcctcttccgaccatcaagcattttatccgtactcctgatgatgcatggttactcaccactgcgatcccngggaaaacagcattccaggtattagaagaatatcctgattcaggtgaaaatattgttgatgcgctggcagtgttcctgcgccggttgcattcgattcctctttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataancttttgccattctcaccggattcagtcgtcactcatggtgatttctcacttgataaccttatttttgaccaggcgaaattaataggttgtattgatcttcgacgagtcggaatcgcagaccgataccaggatcttgccatcctatggaactgcctcggtgagttttctccttcattacagaaacggctttttcaaaaatatggtattgataatcctgatatgaataaattgcagtttcatttgatcctcgatgagtttttctaatcagaattggttaattggttgtaacactggcagagcattacgctgacttgacgggacggcggctttgttgaataaatcgaacttttgctgacttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgagccatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgaaattgtaaacgttaatattttgttaaaattcgcgttaaatatttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcacccaaatcaagttttttgcggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactata

SEQ ID NO: 22 - DNA, 3191 bp, artificial sequence
  plasmid PHP17064
ctagaaatccgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatgatcctatgcgtatggtatgacgtgtgttcaagatgatgacttcaaacctacctatgacgtatggtatgaacgtgtgtcgactgatgacttagatccactcgagcggctataaatacgtacctacgcaccctgcgctaccatccctagagctgcagcttatttttacaacaattaccaacaacaacaaacaacaaacaacattacaattactatttacaattacagtcgacccgtacccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcgcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagttggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgattgcctagaccaaatgttccttgatgcttgttttgtacaatatatataagataatgctgtcctagttgcaggatttggcctgtggtgagcatcatagtctgtagtagttttggtagcaagacattttattttccttttatttaacttactacatgcagtagcatctatctatctctgtagtctgatatctcctgttgtctgtattgtgccgttggattttttgctgtagtgagactgaaaatgatgtgctagtaataatatttctgttagaaatctaagtagagaatctgttgaagaagtcaaaagctaatggaatcaggttacatatcaatgtttttctttttttagcggttggtagacgtgtagattcaacttctcttggagctcacctaggcaatcagtaaaatgcatattccttttttaacttgccatttatttacttttagtggaaattgtgaccaatttgttcatgtagaacggatttggaccattgcgtccacaaaacgtctcttttgctcgatcttcacaaagcgataccgaaatccagagatagttttcaaaagtcagaaatggcaaagttataaatagtaaaacagaatagatgctgtaatcgacttcaataacaagtggcatcacgtttctagtt

SEQ ID NO: 23 - DNA, 2924 bp, artificial sequence
  fragment PHP19340A
cgcgccaagcttggatcctcgaagagaagggttaataacacacttttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccgctgagtgattgctcacgagtgtggtcaccatgccttcagcaagtaccaatgggttgatgatgttgtgggtttgacccttcactcaacacttttagtcccttatttctcatggaaaataagccatcgccgccatcactccaacacaggttcccttgaccgtgatgaagtgtttgtcccaaaaccaaaatccaaagttgcatggttttccaagtacttaaacaaccctctaggaagggctgtttctcttctcgtcacactcacaatagggtggcctatgtatttagccttcaatgtctctggtagaccctatgatagttttgcaagccactaccacccttatgctcccatatattctaaccgtgagaggcttctgatctatgtctctgatgttgctttgttttctgtgacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattaagcggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccgtcgacgg

SEQ ID NO: 24 - DNA, 4511 bp, artificial sequence
  fragment PHP17752
cgcgccaagcttggatcccccctcgaggtcgacggtatcgataagcttctgcaggaattctgagctagcgaagttcctattccgaagttcctattcttcaaaaagtataggaacttcagacgtcctcgagtccgtcctgtagaaaccccaacccgtgaaatcaaaaaactcgacggcctgtgggcattcagtctggatcgcgaaaactgtggaattgatccagaattcgctagcgaagttcctattccgaagttcctattctctagaaagtataggaacttcagatccagaattcggtccgggccatcgtggcctcttgctcttcaggatgaagagctatgtttcgcgccaagcttggatcctagaactagaaacgtgatgccacttgttattgaagtcgattacagcatctattctgttttactatttataactttgccatttctgacttttgaaaactatctctggatttcggtatcgctttgtgaagatcgagcaaaagagacgttttgtggacgcaatggtccaaatccgttctacatgaacaaattggtcacaatttccactaaaagtaaataaatggcaagttaaaaaaggaatatgcattttactgattgcctaggtgagctccaagagaagttgaatctacacgtctaccaaccgctaaaaaaagaaaaacattgatatgtaacctgattccattagcttttgacttcttcaacagattctctacttagatttctaacagaaatattattactagcacatcattttcagtctcactacagcaaaaaatccaacggcacaatacagacaacaggagatatcagactacagagatagatagatgctactgcatgtagtaagttaaataaaaggaaaataaaatgtcttgctaccaaaactactacagactatgatgctcaccacaggccaaatcctgcaactaggacagcattatcttatatatattgtacaaaacaagcatcaaggaacatttggtctaggcaatcagtacctcgttctaccatcaccctcagttatcacatccttgaaggatccattactgggaatcatcggcaacacatgctcctgatggggcacaatgacatcaagaaggtaggggccaggggtgtccaacattctctgaattgccgctctaagctcttccttcttcgtcactcgcgctgccggtatcccacaagcatcagcaaacttgagcatgtttgggaatatctcgctctcgctagacggatctccaagataggtgtgagctctattggacttgtagaacctatcctccaactgaaccaccatacccaaatgctgattgttcaacaacaatatcttaactgggagattctccactcttatagtggccaactcctgaacattcatgatgaaactaccatccccatcaatgtcaaccacaacagccccagggttagcaacagcagcaccaatagccgcaggcaatccaaaacccatggctccaagaccccctgaggtcaaccactgcctcggtctcttgtacttgtaaaactgcgcagcccacatttgatgctgcccaaccccagtactaacaatagcatctccattagtcaactcatcaagaacctcgatagcatgctgcggagaaatcgcgtcctggaatgtcttgtaacccaatggaaacttgtgtttctgcacattaatctcttctctccaacctccaagatcaaacttaccctccactcctttctcctccaaaatcatattaattcccttcaaggccaacttcaaatccgcgcaaaccgacacgtgcgcctgcttgttcttcccaatctcggcagaatcaatatcaatgtgaacaatcttagccctactagcaaaagcctcaagcttcccagtaacacggtcatcaaaccttaccccaaaggcaagcaacaaatcactattgtcaacagcatagttagcataaacagtaccatgcatacccagcatctgaagggaatattcatcaccaataggaaaagttccaagacccattaaagtgctagcaacgggaataccagtgagttcaacaaagcgcctcaattcagcactggaattcaaactgccaccgccgacgtagagaacgggcttttgggcctccatgatgagtctgacaatgtgttccaattgggcctcggcggggggcctgggcagcctggcgaggtaaccggggaggttaacgggctcgtcccaattaggcacggcgagttgctgctgaacgtctttgggaatgtcgatgaggaccggaccggggcggccggaggtggcgacgaagaaagcctcggcgacgacgcgggggatgtcgtcgacgtcgaggatgaggtagttgtgcttcgtgatggatctgctcacctccacgatcggggtttcttggaaggcgtcggtgccgatcatccggcgggcgacctggccggtgatggcgacgactgggacgctgtccattaaagcgtcggcgaggccgctcacgaggttggtggcgccggggccggaggtggcaatgcagacgccggggaggccggaggaacgcgcgtagccttcggcggcgaagacgccgccctgctcgtggcgcgggagcacgttgcggatggcggcggagcgcgtgagcgcctggtggatctccatcgacgcaccgccggggtacgcgaacaccgtcgtcacgccctgcctctccagcgcctccacaaggatgtccgcgcccttgcgaggttcgccggaggcgaaccgtgacacgaagggctccgtggtcggcgcttccttggtgaagggcgccgccgtggggggtttggagatggaacatttgattttgagagcgtggttgggtttggtgagggtttgatgagagagagggagggtggatctagtaatgcgtttggggaaggtggggtgtgaagaggaagaagagaatcgggtggttctggaagcggtggccgccattgtgttgtgtggcatggttatacttcaaaaactgcacaacaagcctagagttagtacctaaacagtaaatttacaacagagagcaaagacacatgcaaaaatttcagccataaaaaaagttataatagaatttaaagcaaaagtttcattttttaaacatatatacaaacaaactggatttgaaggaagggattaattcccctgctcaaagtttgaattcctattgtgacctatactcgaataaaattgaagcctaaggaatgtatgagaaacaagaaaacaaaacaaaactacagacaaacaagtacaattacaaaattcgctaaaattctgtaatcaccaaaccccatctcagtcagcacaaggcccaaggtttattttgaaataaaaaaaaagtgattttatttctcataagctaaaagaaagaaaggcaattatgaaatgatttcgactagatctgaaagtccaacgcgtattccgcagatattaaagaaagagtagagtttcacatggatcctagatggacccagttgaggaaaaagcaaggcaaagcaaaccagaagtgcaagatccgaaattgaaccacggaatctaggatttggtagagggagaagaaaagtaccttgagaggtagaagagaagagaagagcagagagatatatgaacgagtgtgtcttggtctcaactctgaagcgatacgagtttagaggggagcattgagttccaatttatagggaaaccgggtggcaggggtgagttaatgacggaaaagcccctaagtaacgagattggattgtgggttagattcaaccgtttgcatccgcggcttagattggggaagtcagagtgaatctcaaccgttgactgagttgaaaattgaatgtagcaaccaattgagccaaccccagcctttgccctttgattttgatttgtttgttgcatactttttatttgtcttctggttctgactctctttctctcgtttcaatgccaggttgcctactcccacaccactcacaagaagattctactgttagtattaaatattttttaatgtattaaatgatgaatgcttttgtaaacagaacaagactatgtctaataagtgtcttgcaacattttttaagaaattaaaaaaaatatatttattatcaaaatcaaatgtatgaaaaatcatgaataatataattttatacatttttttaaaaaatcttttaatttcttaattaatatcttaaaaataatgattaatatttaacccaaaataattagtatgattggtaaggaagatatccatgttatgtttggatgtgagtttgatctagagcaaagcttactagagtcgaccgatccgtcgacggcgcgcgcgcctctagttgaagacacgttcatgtcttcatcgtaagaagacactcagtagtcttcggccagaatggcccggaccgaagcttctgcaggaattctgagctagcgaagttcctattccgaagttcctattctctagaaagtataggaacttcagatccactaggatccgtcgacgg

SEQ ID NO: 25 - DNA, 5437 bp, artificial sequence
  plasmid  PHP19340
ggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccgtcgacggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccaagcttggatcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccgctgagtgattgctcacgagtgtggtcaccatgccttcagcaagtaccaatgggttgatgatgttgtgggtttgacccttcactcaacacttttagtcccttatttctcatggaaaataagccatcgccgccatcactccaacacaggttcccttgaccgtgatgaagtgtttgtcccaaaaccaaaatccaaagttgcatggttttccaagtacttaaacaaccctctaggaagggctgtttctcttctcgtcacactcacaatagggtggcctatgtatttagccttcaatgtctctggtagaccctatgatagttttgcaagccactaccacccttatgctcccatatattctaaccgtgagaggcttctgatctatgtctctgatgttgctttgttttctgtgacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattaagc

SEQ ID NO: 26 - DNA, 7025 bp, artificial sequence
  plasmid PHP17752
gatccgtcgacggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccaagcttggatcccccctcgaggtcgacggtatcgataagcttctgcaggaattctgagctagcgaagttcctattccgaagttcctattcttcaaaaagtataggaacttcagacgtcctcgagtccgtcctgtagaaaccccaacccgtgaaatcaaaaaactcgacggcctgtgggcattcagtctggatcgcgaaaactgtggaattgatccagaattcgctagcgaagttcctattccgaagttcctattctctagaaagtataggaacttcagatccagaattcggtccgggccatcgtggcctcttgctcttcaggatgaagagctatgtttcgcgccaagcttggatcctagaactagaaacgtgatgccacttgttattgaagtcgattacagcatctattctgttttactatttataactttgccatttctgacttttgaaaactatctctggatttcggtatcgctttgtgaagatcgagcaaaagagacgttttgtggacgcaatggtccaaatccgttctacatgaacaaattggtcacaatttccactaaaagtaaataaatggcaagttaaaaaaggaatatgcattttactgattgcctaggtgagctccaagagaagttgaatctacacgtctaccaaccgctaaaaaaagaaaaacattgatatgtaacctgattccattagcttttgacttcttcaacagattctctacttagatttctaacagaaatattattactagcacatcattttcagtctcactacagcaaaaaatccaacggcacaatacagacaacaggagatatcagactacagagatagatagatgctactgcatgtagtaagttaaataaaaggaaaataaaatgtcttgctaccaaaactactacagactatgatgctcaccacaggccaaatcctgcaactaggacagcattatcttatatatattgtacaaaacaagcatcaaggaacatttggtctaggcaatcagtacctcgttctaccatcaccctcagttatcacatccttgaaggatccattactgggaatcatcggcaacacatgctcctgatggggcacaatgacatcaagaaggtaggggccaggggtgtccaacattctctgaattgccgctctaagctcttccttcttcgtcactcgcgctgccggtatcccacaagcatcagcaaacttgagcatgtttgggaatatctcgctctcgctagacggatctccaagataggtgtgagctctattggacttgtagaacctatcctccaactgaaccaccatacccaaatgctgattgttcaacaacaatatcttaactgggagattctccactcttatagtggccaactcctgaacattcatgatgaaactaccatccccatcaatgtcaaccacaacagccccagggttagcaacagcagcaccaatagccgcaggcaatccaaaacccatggctccaagaccccctgaggtcaaccactgcctcggtctcttgtacttgtaaaactgcgcagcccacatttgatgctgcccaaccccagtactaacaatagcatctccattagtcaactcatcaagaacctcgatagcatgctgcggagaaatcgcgtcctggaatgtcttgtaacccaatggaaacttgtgtttctgcacattaatctcttctctccaacctccaagatcaaacttaccctccactcctttctcctccaaaatcatattaattcccttcaaggccaacttcaaatccgcgcaaaccgacacgtgcgcctgcttgttcttcccaatctcggcagaatcaatatcaatgtgaacaatcttagccctactagcaaaagcctcaagcttcccagtaacacggtcatcaaaccttaccccaaaggcaagcaacaaatcactattgtcaacagcatagttagcataaacagtaccatgcatacccagcatctgaagggaatattcatcaccaataggaaaagttccaagacccattaaagtgctagcaacgggaataccagtgagttcaacaaagcgcctcaattcagcactggaattcaaactgccaccgccgacgtagagaacgggcttttgggcctccatgatgagtctgacaatgtgttccaattgggcctcggcggggggcctgggcagcctggcgaggtaaccggggaggttaacgggctcgtcccaattaggcacggcgagttgctgctgaacgtctttgggaatgtcgatgaggaccggaccggggcggccggaggtggcgacgaagaaagcctcggcgacgacgcgggggatgtcgtcgacgtcgaggatgaggtagttgtgcttcgtgatggatctgctcacctccacgatcggggtttcttggaaggcgtcggtgccgatcatccggcgggcgacctggccggtgatggcgacgactgggacgctgtccattaaagcgtcggcgaggccgctcacgaggttggtggcgccggggccggaggtggcaatgcagacgccggggaggccggaggaacgcgcgtagccttcggcggcgaagacgccgccctgctcgtggcgcgggagcacgttgcggatggcggcggagcgcgtgagcgcctggtggatctccatcgacgcaccgccggggtacgcgaacaccgtcgtcacgccctgcctctccagcgcctccacaaggatgtccgcgcccttgcgaggttcgccggaggcgaaccgtgacacgaagggctccgtggtcggcgcttccttggtgaagggcgccgccgtggggggtttggagatggaacatttgattttgagagcgtggttgggtttggtgagggtttgatgagagagagggagggtggatctagtaatgcgtttggggaaggtggggtgtgaagaggaagaagagaatcgggtggttctggaagcggtggccgccattgtgttgtgtggcatggttatacttcaaaaactgcacaacaagcctagagttagtacctaaacagtaaatttacaacagagagcaaagacacatgcaaaaatttcagccataaaaaaagttataatagaatttaaagcaaaagtttcattttttaaacatatatacaaacaaactggatttgaaggaagggattaattcccctgctcaaagtttgaattcctattgtgacctatactcgaataaaattgaagcctaaggaatgtatgagaaacaagaaaacaaaacaaaactacagacaaacaagtacaattacaaaattcgctaaaattctgtaatcaccaaaccccatctcagtcagcacaaggcccaaggtttattttgaaataaaaaaaaagtgattttatttctcataagctaaaagaaagaaaggcaattatgaaatgatttcgactagatctgaaagtccaacgcgtattccgcagatattaaagaaagagtagagtttcacatggatcctagatggacccagttgaggaaaaagcaaggcaaagcaaaccagaagtgcaagatccgaaattgaaccacggaatctaggatttggtagagggagaagaaaagtaccttgagaggtagaagagaagagaagagcagagagatatatgaacgagtgtgtcttggtctcaactctgaagcgatacgagtttagaggggagcattgagttccaatttatagggaaaccgggtggcaggggtgagttaatgacggaaaagcccctaagtaacgagattggattgtgggttagattcaaccgtttgcatccgcggcttagattggggaagtcagagtgaatctcaaccgttgactgagttgaaaattgaatgtagcaaccaattgagccaaccccagcctttgccctttgattttgatttgtttgttgcatactttttatttgtcttctggttctgactctctttctctcgtttcaatgccaggttgcctactcccacaccactcacaagaagattctactgttagtattaaatattttttaatgtattaaatgatgaatgcttttgtaaacagaacaagactatgtctaataagtgtcttgcaacattttttaagaaattaaaaaaaatatatttattatcaaaatcaaatgtatgaaaaatcatgaataatataattttatacatttttttaaaaaatcttttaatttcttaattaatatcttaaaaataatgattaatatttaacccaaaataattagtatgattggtaaggaagatatccatgttatgtttggatgtgagtttgatctagagcaaagcttactagagtcgaccgatccgtcgacggcgcgcgcgcctctagttgaagacacgttcatgtcttcatcgtaagaagacactcagtagtcttcggccagaatggcccggaccgaagcttctgcaggaattctgagctagcgaagttcctattccgaagttcctattctctagaaagtataggaacttcagatccactag